(19) United States
(12) Patent Application Publication
(10) Pub. No.: US 2013/0035721 A1
(43) Pub. Date: Feb. 7, 2013

(54) SURGICAL ANCHOR

(71) Applicant: John Eric Brunelle, Carlsbad, CA (US)

(72) Inventor: John Eric Brunelle, Carlsbad, CA (US)

(21) Appl. No.: 13/198,534

(22) Filed: Aug. 4, 2011

(51) Int. Cl. A61B 17/04

(52) U.S. Cl. 606/232

(57) ABSTRACT

Surgical anchors can be utilized to approximate and hold soft tissue in, at, or near a boney insertion site. In some examples, the surgical anchors include a receiving body and a fixation member such as, for example, a fixation screw. The receiving body may include an elongated connecting member and an aperture defined at a distal end of the elongated connecting member. The aperture may be configured to receive a surgical attachment element. Depending on the configuration, the receiving body may be connected to the fixation member via the elongated connecting member such that the fixation member can move relative to the receiving body. Movement of the fixation member relative to the receiving body may allow a user to control and adjust the amount of tension placed on the surgical attachment element, which may enhance the mechanics of a repair operation.

### TECHNICAL FIELD

This disclosure relates to surgical anchors and, more particularly, to implantable surgical anchors.

### BACKGROUND

Surgical repair of damaged soft tissue is a procedure that is being carried out with increasing frequency. One of the most common methods for repair of soft tissue damage at or near a boney insertion site involves the approximation and reattachment of the damaged soft tissue to the insertion site. Typically, a suture is connected to the damaged soft tissue at one end and then affixed to the insertion site using bone tunnels or a suture anchor at the other end. In some examples, a suture anchor is first engaged with bone at the soft tissue insertion site and a suture is subsequently used to approximate and secure the damaged soft tissue.

In practice, ensuring that a suture anchor holds the soft tissue being secured in a proper position and under an appropriate amount of tension can help facilitate proper tissue reattachment and eventual healing. For example, with some types of tissue, ensuring that there is an appropriate distribution of tension around the tissue can help minimize disruption of the tissue-bone interface, leading to improved nutrient flow and tissue healing. Conversely, an excessive amount of tension around the tissue can cause further mechanical damage and impede biological healing mechanisms leading to sub-optimal clinical outcomes. For these reasons, ensuring adequate tensioning and positioning of tissue relative to a boney insertion site may be useful to produce an efficacious clinical outcome.

### SUMMARY

In general, this disclosure is directed towards implantable surgical anchors for securing tissue to a bone structure. In some examples, the surgical anchors include a fixation member that is configured to mechanically engage with the bone structure and a receiving body that receives and places a surgical attachment element. In some examples, the fixation member can move independently of, and thus be repositionable relative to, the receiving body. Accordingly, in these examples, a surgeon can use the receiving body to adjust the amount of tension placed on the surgical attachment element and independently position the fixation member relative to the receiving body to achieve fixation of the surgical attachment element. If the desired tension on the surgical attachment element is not achieved following initial fixation, the surgeon can reversibly position the fixation member relative to the receiving body to remove the surgical anchor from the bony insertion site. Once removed, the surgeon can further adjust tension on the surgical attachment element and then re-position the fixation member relative to the receiving body to re-establish fixation.

In one example according to the disclosure, a surgical anchor is described that includes a fixation member that includes an anchor body extending between a proximal end and a distal end, and a receiving body that includes an elongated connecting member extending between a proximal end and a distal end, the receiving body including an aperture defined at the distal end of the elongated connecting member that is configured to receive a surgical attachment element. According to the example, the receiving body is connected to the fixation member via the elongated connecting member, and the fixation member is configured to move relative to the receiving body.

In another example, a surgical anchor system is described that includes an anchor inserter and a surgical anchor. The anchor inserter includes a shaft defining a lumen extending from a proximal end of the shaft to a distal end of the shaft, and a rod extending through the lumen defined by the shaft. The surgical anchor includes a fixation member that includes an anchor body extending between a proximal end and a distal end, and a receiving body that includes an elongated connecting member extending between a proximal end and a distal end, the receiving body including an aperture defined at the distal end of the elongated connecting member that is configured to receive a surgical attachment element. According to the example, the fixation member is connected at the distal end of the shaft, the receiving body is connected at a distal end of the rod, and the receiving body is connected to the fixation member via the elongated connecting member so that moving the shaft relative to the rod moves the fixation member relative to the receiving body.

In another example, a method is described that includes inserting a surgical attachment element through an aperture defined by a receiving body, where the receiving body includes an elongated connecting member extending between a proximal end to a distal end, and the aperture is defined at the distal end of the elongated connecting member. According to the example, the method also includes moving a fixation member that includes an anchor body along the elongated connecting member so as to vary a distance between a distal end of the fixation member and the aperture of the receiving body.

In another example, a surgical anchor includes receiving means for receiving a surgical attachment element, and fixation means for securing the surgical attachment element to a bone hole, where the receiving means are connected to the fixation means, and the fixation means are configured to move linearly along an axis of the receiving means.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A-1C are schematic illustrations of an example surgical anchor in accordance with the disclosure.

FIGS. 2A-2F are schematic illustrations of another example surgical anchor in accordance with the disclosure.

FIGS. 3A-3C are schematic illustrations of another example surgical anchor in accordance with the disclosure.

FIG. 4 is an illustration of an example anchor inserter that can be used to insert the surgical anchor of FIGS. 1A-1C, FIGS. 2A-2F, or FIGS. 3A-3C.

FIGS. 5A-5C are enlarged views of the example surgical anchor of FIGS. 1A-1C connected to the anchor inserter of FIG. 4.

FIGS. 6A and 6B are drawings of example corresponding connection features that can be used to connect a surgical anchor to the anchor inserter of FIG. 4.

FIG. 7 is a cross-sectional illustration of additional example corresponding connection features that can be used to connect a surgical anchor to the anchor inserter of FIG. 4.

FIG. 8 is a schematic illustration of an example surgical anchor and anchor inserter kit.

FIGS. 9A-9C are conceptual illustrations of example surgical tools that may be used with a surgical anchor.

FIG. 10 is a conceptual illustration of an example piece of soft tissue connected to a suture.

FIG. 11 is a conceptual illustration of an example piece of soft tissue connected to a surgical mesh.

FIGS. 12-21 are conceptual illustrations of example steps that may be performed to anchor the example pieces of soft tissue of FIGS. 10 and 11 to a bone structure using the example surgical anchor of FIGS. 1A-1C, FIGS. 2A-2F, or FIGS. 3A-3C.

### DETAILED DESCRIPTION

This disclosure relates to surgical anchors that are typically used for anchoring a surgical attachment element to bone during surgery. The surgical attachment element can be a composite implantable tissue attachment device (which may be referred to as a surgical mesh attachment device), a suture, a portion of soft tissue (e.g., a ruptured host tissue, an autograft, an allograft, or a xenograft tissue), or another similar element that is intended to be attached to bone during surgery. In examples where the surgical attachment element is a surgical mesh attachment device or a suture, the surgical attachment element may further be connected to a portion of soft tissue. Thus, the surgical anchors described herein can be used to secure soft tissue to bone or a bone-like structure during a surgical procedure.

Depending on the configuration, the surgical anchor described in this disclosure may be used to controllably adjust the amount of tension placed on the surgical attachment element during surgery. For instance, in some examples, the surgical anchor includes a receiving body that receives the surgical attachment element, and a fixation member that is configured to mechanically engage with a bone structure and secure the surgical attachment element. The fixation member can move independently of, and thus be repositionable relative to, the receiving body. Accordingly, a surgeon or other health care practitioner (collectively referred to herein as “a surgeon”) can manipulate the receiving body, allowing the surgeon to control the amount of tension applied to the surgical attachment element during placement within a bone structure. Thereafter, the surgeon can deploy the fixation member to both mechanically engage and secure the surgical attachment element to the bone structure.

Depending on the specific design of the surgical anchor, after the fixation member is secured to the bone structure, the surgeon may be able to remove the fixation member from the bone structure and re-adjust the tension applied to the surgical attachment element. In contrast to other types of surgical anchors that do not permit controlled removal, re-tensioning and repositioning after deployment (e.g., fixation), the surgical anchor of the present disclosure may be used to control the tension and/or positioning of soft tissue being anchored after the anchor has been secured. Better tensioning and positioning capability may allow the surgeon to more effectively manipulate the soft tissue to optimize repair mechanics specific to a particular injury or physiological function.

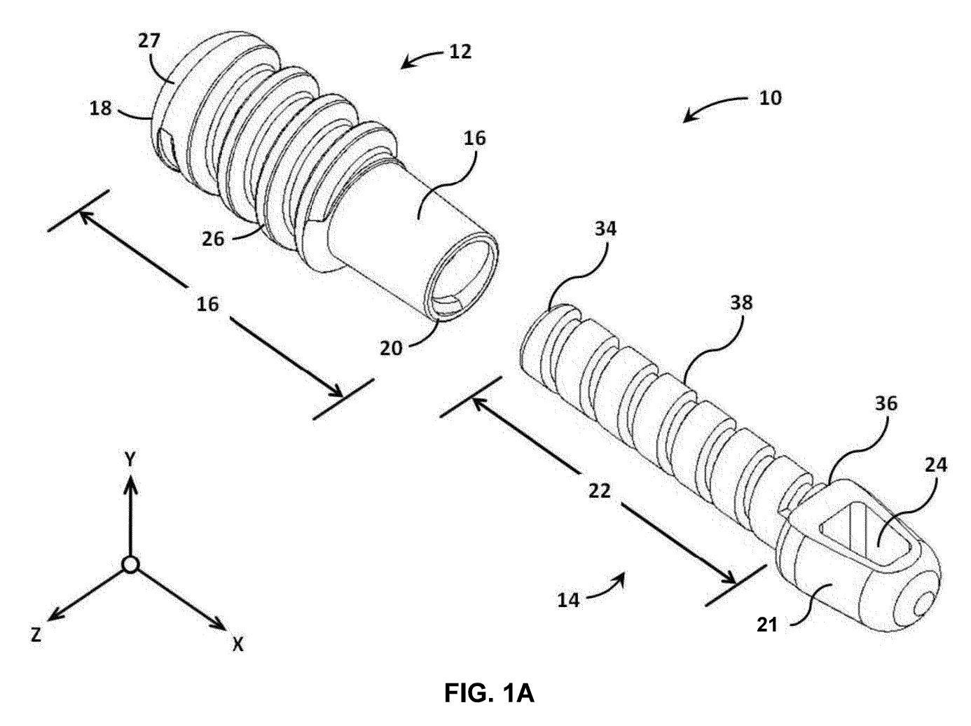

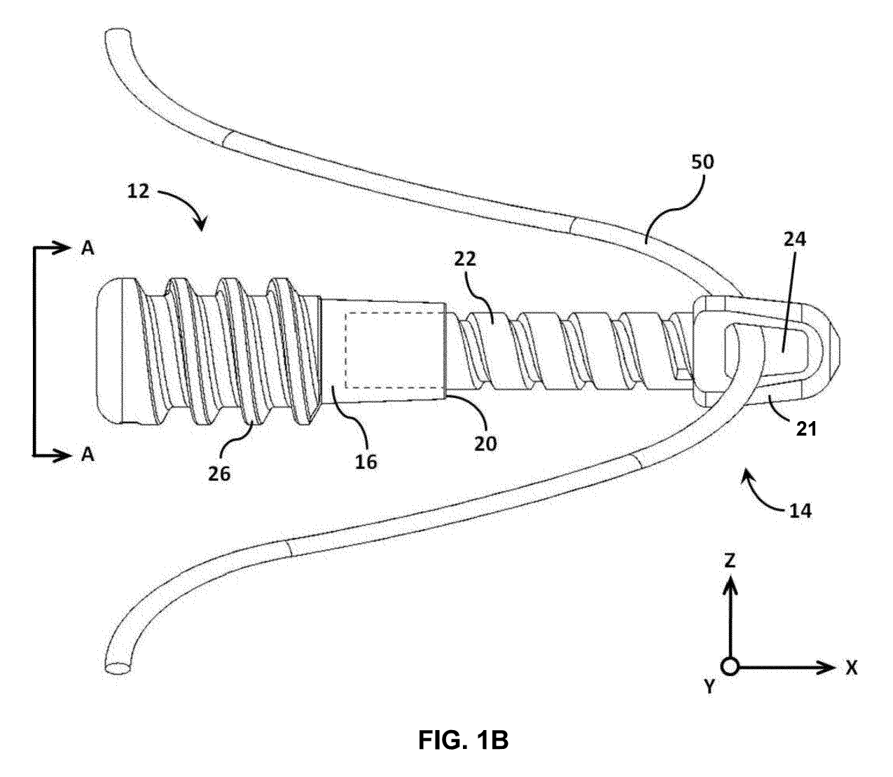

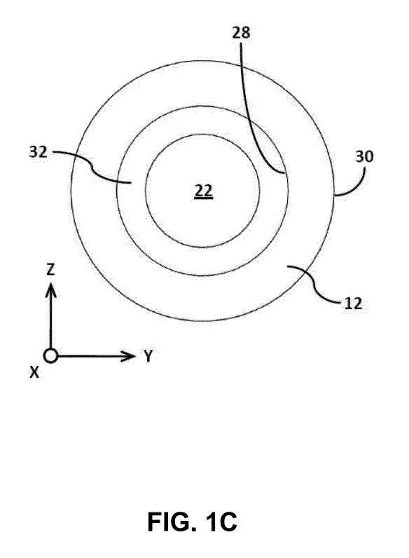

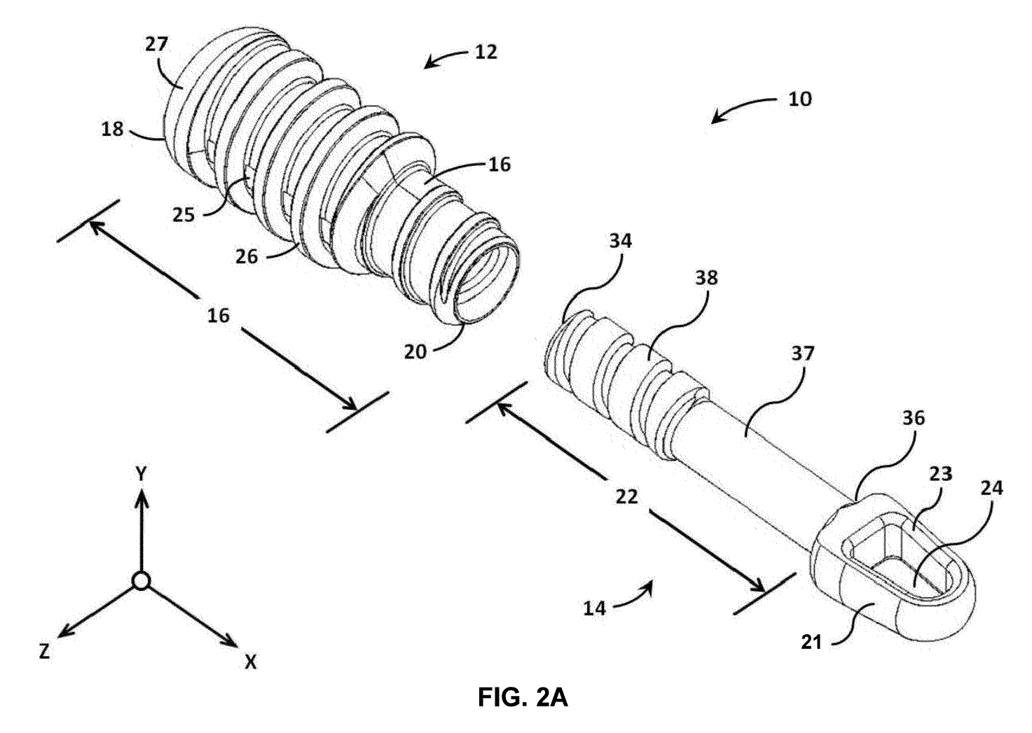

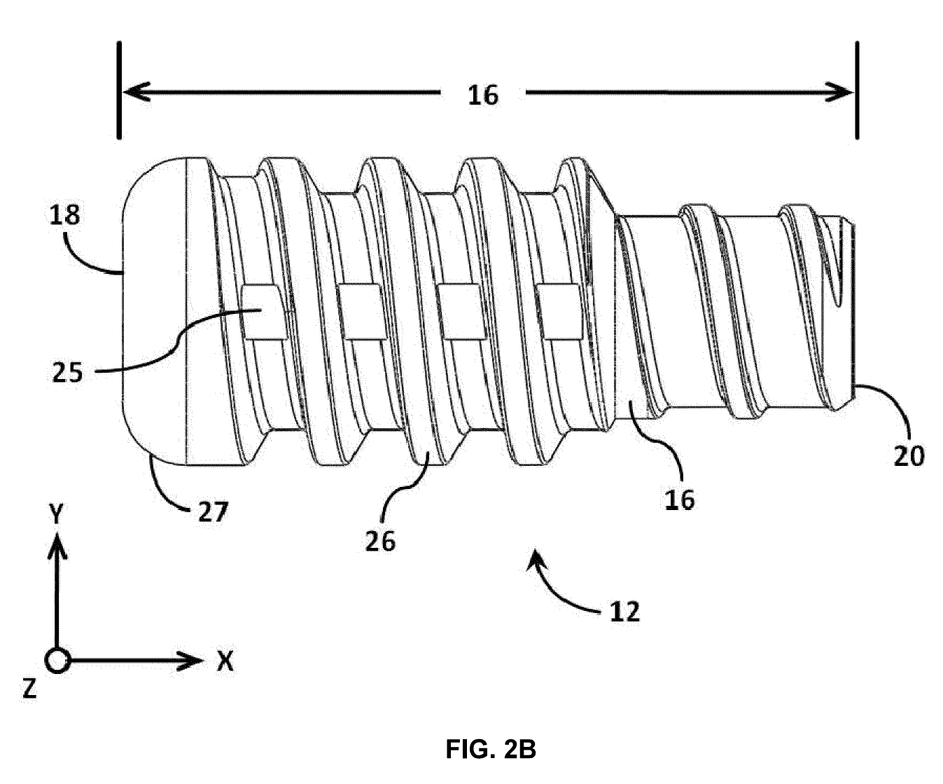

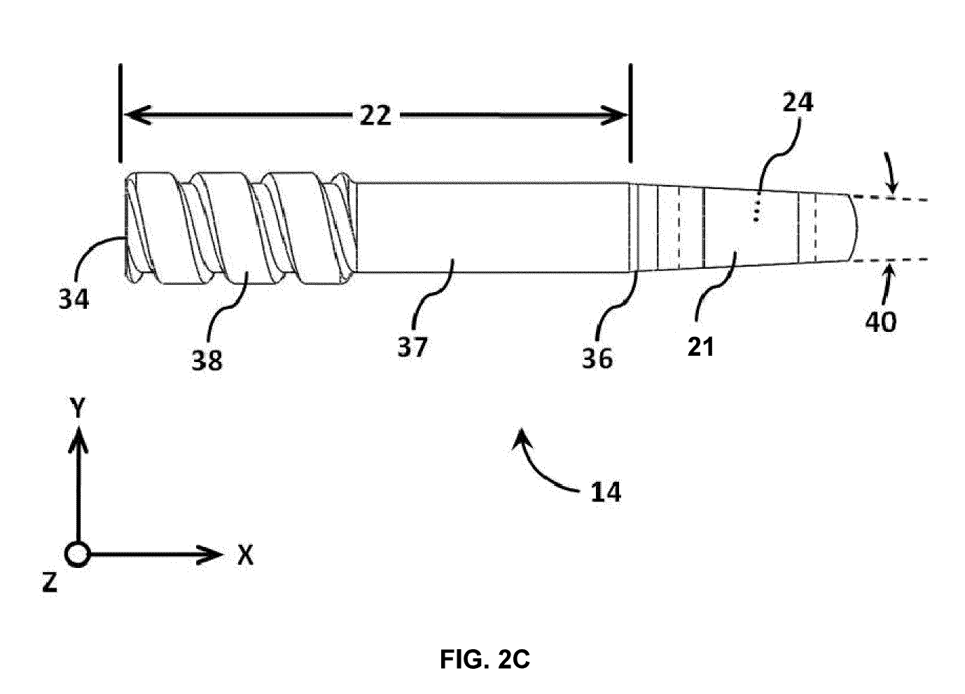

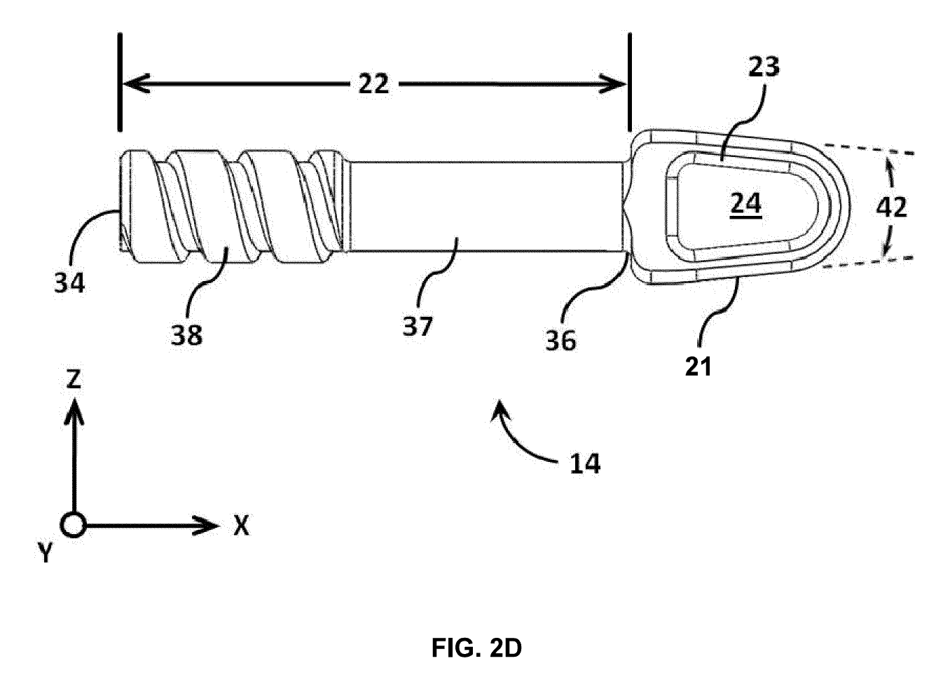

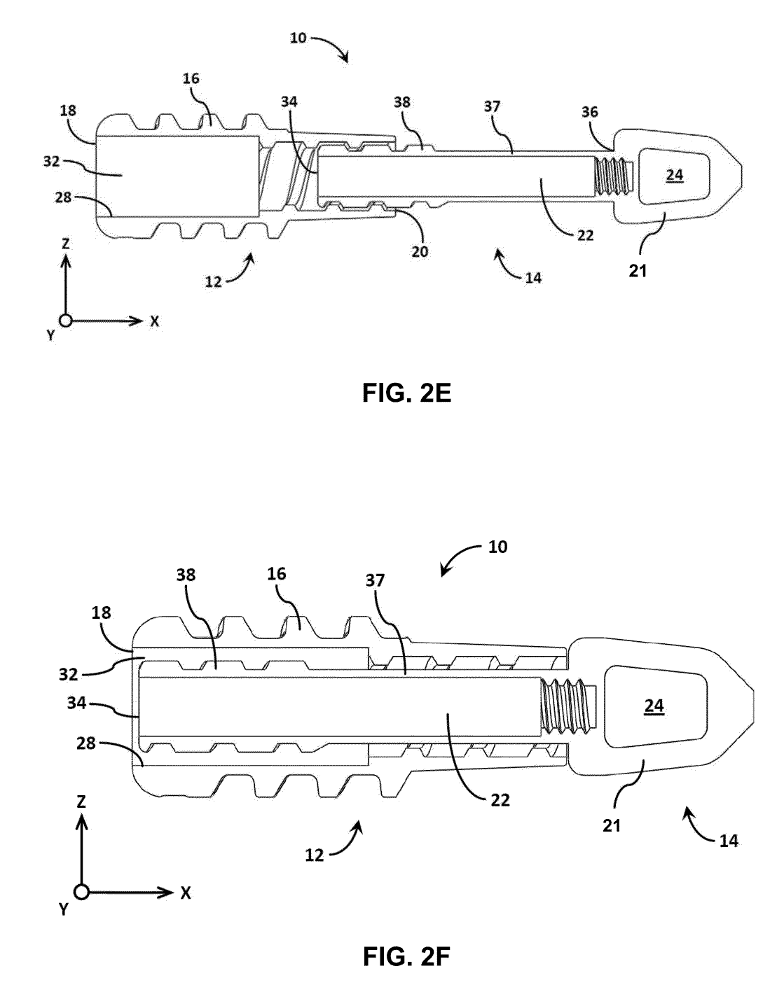

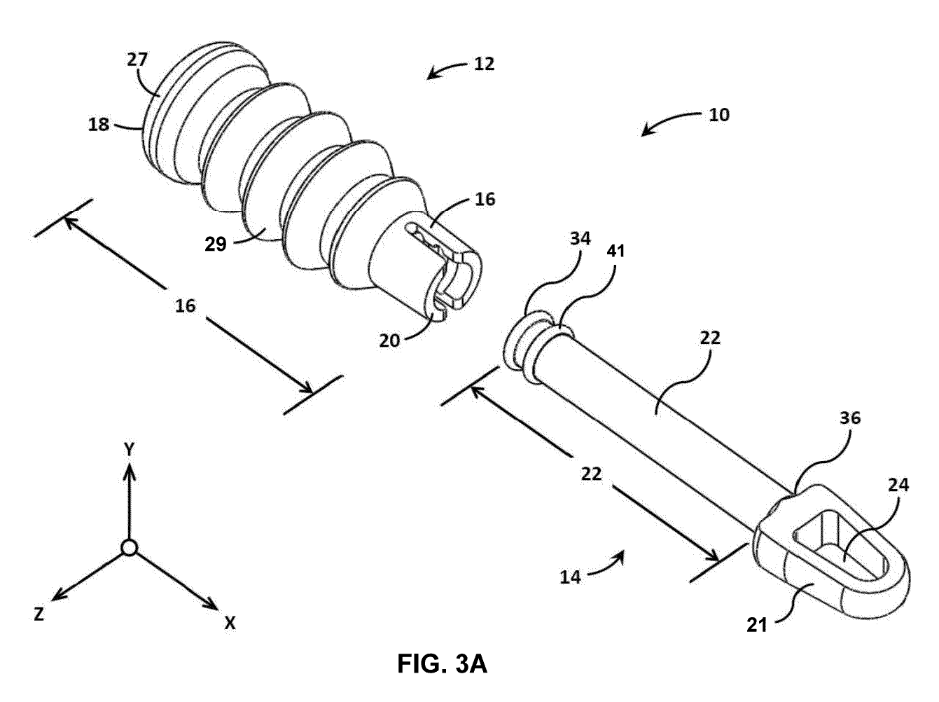

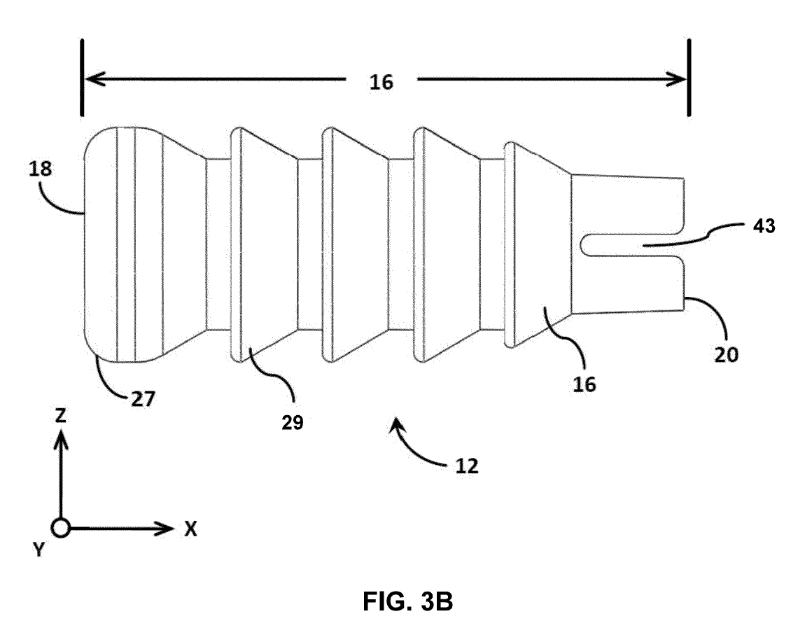

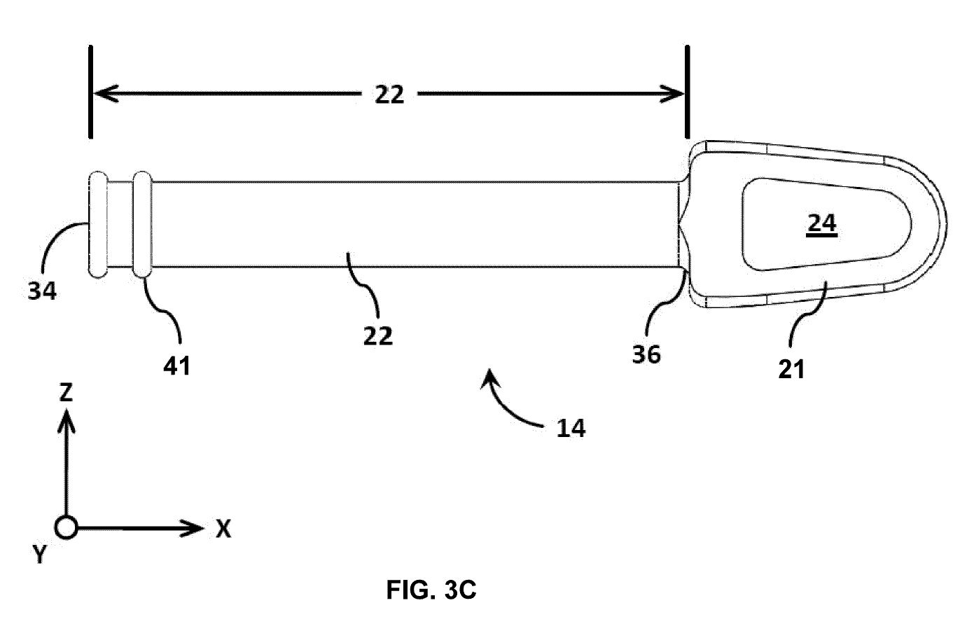

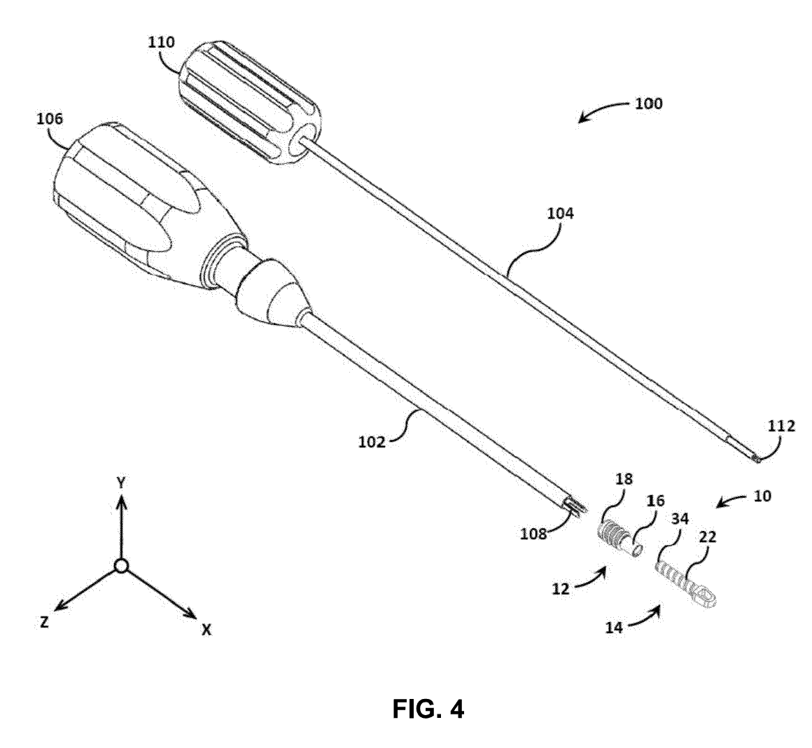

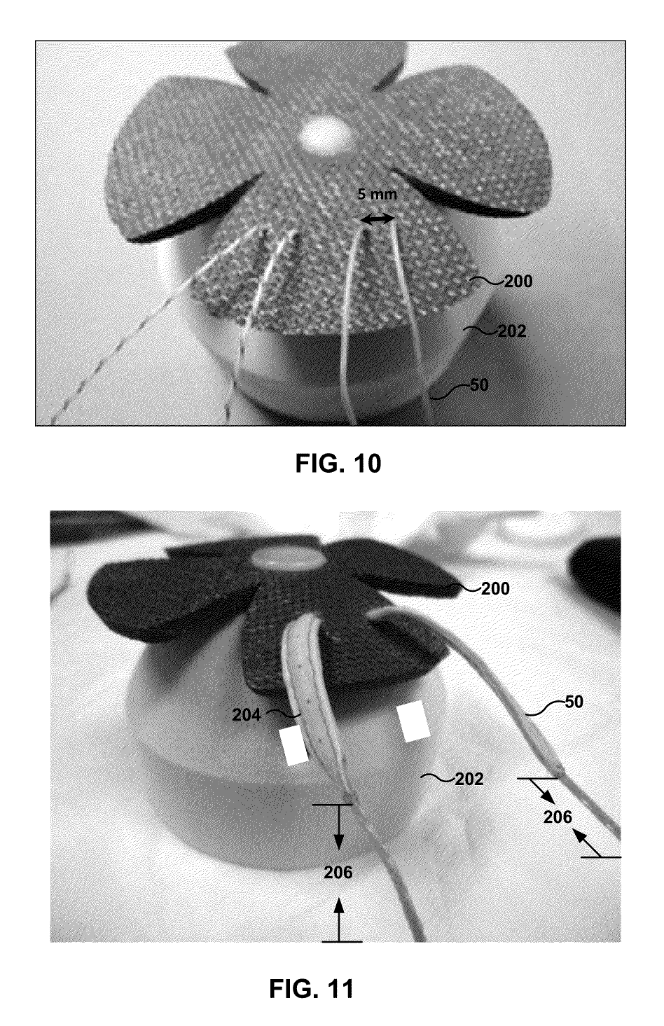

An example surgical anchor system that includes an example anchor inserter, an example surgical anchor, and associated example auxiliary instrumentation will be described in greater detail with reference to FIGS. 4-9. Further, an example method for securing tissue to bone will be described with reference to FIGS. 10-21. However, example surgical anchors according to the disclosure will first be described with reference to FIGS. 1A-1C, FIGS. 2A-2F, or FIGS. 3A-3C.

FIGS. 1A, 2A, and 3A are schematic illustrations of different configurations of an example surgical anchor 10, which includes a fixation member 12 and a receiving body 14. Fixation member 12 includes an anchor body 16 that extends from a proximal end 18 to a distal end 20. Receiving body 14 includes an elongated connecting member 22 and a portion 21 that defines an aperture 24. Receiving body 14 can be connected to fixation member 12 by inserting elongated connecting member 22 into distal end 20 of fixation member 12 (e.g., as illustrated in FIG. 1B). Distal end 20 of fixation member 12 may include one or more mechanical attachment elements for mechanically connecting elongated connecting member 22 to fixation member 12. Surgical anchor 10 can include additional or different features, as described in greater detail below.

Once assembled, fixation member 12 is configured to move relative to receiving body 14 so that aperture 24 can be controllably positioned relative to distal end 20 of fixation member 12, e.g., at a boney insertion site prior to deployment of fixation member 12. Specifically, in the examples of FIGS. 1A, 2A, and 3A, fixation member 12 is configured to translate axially along the major axis defined by elongated connecting member 22 so that the position of the distal end 20 of anchor body 16 moves relative to the position of aperture 24.

During surgery, surgical anchor 10 can be secured to a bone or bone-like structure (collectively referred to herein as “a bone structure”) by inserting the portion 21 of surgical anchor 10 defining aperture 24 into a bone structure. In some examples, portion 21 of surgical anchor 10 defining aperture 24 is inserted into a pre-drilled or otherwise preformed hole defined in the bone structure. In other examples, such as examples in which the bone structure exhibits a comparatively low density, the portion defining aperture 24 may be inserted directly into the bone structure without first preforming a hole. Prior to inserting the surgical anchor into the bone structure in either set of examples, a surgical attachment element 50 (FIG. 1B) may be passed through aperture 24 so that one end of the surgical attachment element resides on one side of the aperture and another end of the surgical attachment element resides on another side of the aperture.

With the portion of surgical anchor 10 defining aperture 24 inserted into a hole in a bone structure, fixation member 12 can be translated along elongated connecting member 22 (e.g., in the X-direction indicated on FIGS. 1A, 2A, and 3A) to secure surgical anchor 10 and surgical attachment element 50 extending through aperture 24 to the bone structure. In particular, fixation member 12 can be translated along elongated connecting member 22 until the fixation member mechanically (e.g., frictionally) engages with a sidewall of the hole in the bone structure. This mechanical engagement can secure fixation member 12 and, hence, surgical anchor 10, to the bone structure.

As described in greater detail below, fixation member 12 can move independently of receiving body 14. For example, in some configurations, fixation member 12 can translate proximally and distally relative to receiving body 14 (i.e., in the positive X-direction and negative X-direction in the examples of FIGS. 1A, 2A, and 3A). This translational movement can allow a surgeon to adjust the tensioning and/or positioning of surgical attachment element 50 prior to deploying fixation member 12 to secure the fixation member into a bone structure. Such relative movement between fixation member 12 and receiving body 14 can also allow the surgeon to reversibly move fixation member 12 relative to aperture 24 so as to disengage the fixation member from the bone structure after deployment. Once disengaged, the surgeon can readjust the tensioning and/or positioning of surgical attachment element 50, e.g., by removing the portion of receiving body 14 defining aperture 24 from the bone hole and thereafter readjusting the tensioning and/or positioning, or by readjusting the tensioning and/or positioning while aperture 24 remains in the bone hole. In either example, after readjusting the tensioning and/or positioning, the surgeon can then re-position fixation member 12 relative to aperture 24, e.g., to re-engage the bone structure and re-establish fixation of the surgical attachment element.

Surgical anchor 10 in the examples of FIGS. 1A, 2A, and 3A includes fixation member 12. Fixation member 12 functions to secure surgical anchor 10 and, accordingly, a surgical attachment element extending through aperture 24 of portion 21, to a bone structure at a fixation location. To facilitate fixation, fixation member 12 can include one or more features for mechanically (e.g., frictionally) engaging with a sidewall of a bone hole. In the example of FIGS. 1A and 1B and FIGS. 2A and 2B, fixation member 12 includes threading 26 extending along at least a portion of anchor body 16 between proximal end 18 and distal end 20 of the anchor body. Threading 26 may be defined as a continuous or discontinuous ridge (e.g., helical ridge) extending outwardly (e.g., in the Y-Z plane indicated on FIGS. 1A and 2A) from anchor body 16. In some examples, the distal portion of threading 26 may be configured to cut threads into an untapped hole as anchor body 16 is driven into the hole. Accordingly, in different examples where fixation member 12 includes threading 26, the fixation member may be referred to as a screw-type fixation member or a self-tapping screw-type fixation member.

Threading 26 engages with a sidewall of a bone structure when fixation member 12 is inserted into the bone structure. In particular, in the example of FIGS. 1A and 2A, threading 26 engages with a sidewall of a hole defined in a bone structure when distal end 20 of fixation member 12 is inserted into the hole. Rotating anchor body 16 clockwise (or counter clockwise in different examples) causes the fixation member to advance distally into the bone hole, thereby securing surgical anchor 10 to a bone structure.

In examples where fixation member 12 includes threading 26, the threading can have a variety of different configurations. In some examples, threading 26 includes rounded edges. Threading with rounded edges can reduce damage to a surgical attachment element during fixation as compared to threading with sharp edges. In some examples, threading 26 extends along substantially the entire length of anchor body 16 from proximal end 18 to distal end 20. An example of such threading is illustrated in FIGS. 2A and 2B, where threading 26 extends along substantially the entire length of anchor body 16 from proximal end 18 to distal end 20. By comparison, threading 26 in the example of FIGS. 1A and 1B extend along less than the entire length of anchor body 16. Increasing the length of threading on fixation member 12 may provide increased mechanical engagement of distal end 20 at the entrance of a bone structure to facilitate initial advancement of fixation member 12. Increasing the length of threading on fixation member 12 may also increase the force required to pull the fixation member out of a bone structure after the fixation member has been fully advanced into the bone structure.

In alternative examples, such as the example of FIGS. 3A and 3B, fixation member 12 includes plug features instead of threading 26. Plug features may include a plurality of separated annular ribs and grooves, a plurality of barbs, or other features that extend outwardly from anchor body 16 and that are discontinuous along the major length. In the example of FIGS. 3A and 3B, fixation member 12 includes a plurality of ribs 29 positioned between a plurality of alternating grooves. Ribs 29 extend outwardly from anchor body 16 in the Y-Z plane indicated on FIGS. 3A and 3B and are discontinuous along the major length of anchor body 16 (e.g., in the X-direction indicated on FIGS. 3A and 3B). When fixation member 12 includes plug features, the fixation member may be impacted into a hole in a bone structure (e.g., forcibly pounded or driven into the hole) rather than screwably inserted into the hole.

While fixation member 12 may include plug features in addition to or in lieu of threading 26, a fixation member with threading may be useful in that the fixation member can be removed (e.g., withdrawn) from a bone structure after being inserted with comparatively little damage to the bone structure. For instance, if a surgeon inserts a threaded fixation member into a bone structure and determines that the fixation member needs to be subsequently withdrawn from the structure, for example because the bone structure is too soft to support the anchor or because the tension placed on the surgical attachment element being fixated is not appropriate, the surgeon can rotate the fixation member counter clockwise (or clockwise depending on the particular configuration) to extract the anchor. By contrast, if the fixation member includes plug features that are forcibly driven into a hole in a bone structure, it may be more difficult for the surgeon to extract the fixation member without damaging the bone structure and/or the surgical attachment element.

Independent of the specific features that fixation member 12 includes for mechanically engaging with a sidewall of a bone hole, the features may prevent the fixation member from pulling out of the bone hole without user assistance. This pullout resistance can be characterized in a non-surgical environment by inserting fixation member 12 into a standardized synthetic polymer foam block until proximal end 18 of the fixation member is flush with the foam block. Thereafter, the axial pullout force—that is, the force required by pull the fixation member out of the foam in the negative X-direction indicated on FIG. 1A—can be measured. In some examples, a fixation member in accordance with the disclosure exhibits an axial pullout force greater than 100N such as, e.g., an axial pullout force greater than 200N, or an axial pullout force greater than 400N. The foregoing axial pullout forces are merely examples, however, and it should be appreciated that the disclosure is not limited in this respect.

Fixation member 12 extends from proximal end 18 to distal end 20. In different examples, fixation member 12 may include a proximal end 18 and/or a distal end 20 that defines a sharp edge where the fixation member transitions from the X-Y to the Y-Z planes, or fixation member 12 may include a proximal end 18 and/or a distal end 20 that defines a rounded edge where the fixation member transitions from the X-Y to the Y-Z planes. In the examples of FIGS. 1A, 2A, and 3A, fixation member 12 defines a rounded proximal end 27. Rounded proximal end 27 may be substantially free of shape edges or corners.

In examples in which fixation member 12 includes rounded proximal end 27, the rounded proximal end may reduce damage to surgical attachment element 50 during and/or after anchor installation into a bone structure as compared to a fixation member that include a proximal with sharp edges or corners. For example, rounded proximal end 27 may reduce abrasion and shearing between surgical attachment element 50 and surgical anchor 10 as compared to when the proximal end of fixation member 12 includes sharp edges or corners. This may be especially true when surgical attachment element 50 is cyclically loaded and unloaded with force, e.g., as may be experienced during injury rehabilitation. In addition, rounded proximal end 27 may increase the fixation of surgical attachment element 50 to a bone structure by applying atraumatic compressive forces to compress surgical attachment element 50 against a wall of a bone hole. While rounded proximal end 27 may increase fixation in different types of bone structures, the fixation increase may be more pronounced when a bone structure includes a layer of cortical bone instead of other types of bone structures.

In some examples, surgical anchor 10 includes one or more apertures to facilitate bone in-growth after the anchor is secured to a bone structure. For instance, in the example of FIGS. 2A and 2B, surgical anchor 10 includes bone in-growth apertures 25. Bone in-growth apertures 25 are arranged along anchor body 16 and are substantially transverse to the major axis of fixation member 12. After implantation, patient bone may grow around fixation member 12, within bone in-growth apertures 25, and/or though out the lumen of fixation member 12, helping to secure the surgical anchor to the bone structure for long-term stability.

Surgical anchor 10 in the examples of FIGS. 1A, 2A, and 3A includes receiving body 14. Receiving body 14 receives surgical attachment element 50 through aperture 24 in order to anchor the surgical attachment element to a bone structure. Receiving body 14 is configured to connect to fixation member 12 so that the fixation member can move with respect to the receiving body. Accordingly, fixation member 12 and receiving body 14 can include any suitable features for mating receiving body 14 to fixation member 12.

In the example configurations of FIGS. 1A, 2A, and 3A, receiving body 14 is configured to mate with fixation member 12 by inserting elongated connecting member 22 into a lumen defined through fixation member 12. Accordingly, in these examples, fixation member 12 defines a lumen extending from proximal end 18 to distal end 20 of anchor body 16 (i.e., in the X-direction indicated on FIGS. 1A, 2A, and 3A). The lumen defined by fixation member 12 is configured (e.g., sized and shaped) to receive elongated connecting member 22. When elongated connecting member 22 of receiving body 14 is inserted into the lumen defined by fixation member 12 (e.g., as illustrated in FIG. 1B) the lumen is closed at distal end 20 of anchor body 16 such that the anchor body no longer defines an open lumen extending through the anchor body.

FIG. 1C is a cross-sectional schematic illustration of surgical anchor 10 taken along the A-A line indicated on FIG. 1B. As seen in this example, fixation member 12 defines an interior surface 28, an exterior surface 30, and a lumen 32. A proximal end of elongated connecting member 22 is inserted into lumen 32 of fixation member 12 so as to connect fixation member 12 to receiving body 14. In some examples, as discussed in greater detail below, fixation member 12 and/or elongated connecting member 22 includes mechanical attachment features (e.g., slots, grooves, threading, or the like which are not illustrated in FIG. 1C) for mechanically connecting fixation member 12 to elongated connecting member 22.

When fixation member 12 is mated to receiving body 14 as illustrated in FIGS. 1B and 1C, fixation member 12 is moveably affixed to aperture 24 via elongated connecting member 22. In such examples, anchor body 16 of fixation member 12 is connected to, but displaced from, aperture 24 of receiving body 14. As discussed in greater detail below, a surgical anchor with such an example arrangement may allow a surgeon to controllably position and tension a surgical attachment element prior to fixating the surgical attachment element to bone. In contrast to anchors that do not include a fixation member mechanically and moveably connected to an aperture, surgical anchors according to some examples of the present disclosure may allow an anchor aperture (e.g., through which a surgical attachment element is passed) to be controllably positioned before the fixation member is fixated to bone at an attachment location.

Receiving body 14 in the examples of FIGS. 1A, 2A, and 3A includes elongated connecting member 22. Elongated connecting member 22 connects aperture 24 to fixation member 12 when receiving body 14 and fixation member 12 are connected as shown in FIG. 1B. Elongated connecting member 22 extends from proximal end 34 to distal end 36, with aperture 24 defined by portion 21 at distal end 36 of the elongated connecting member. In some examples, elongated connecting member 22 defines a length (i.e., in the X-direction indicated on FIGS. 1A, 2A, and 3A) that is greater than a major width (i.e., in Y-Z plane of FIGS. 1A, 2A, and 3A). For instance, in various examples, elongated connecting member 22 may be a pin, rod, shaft, column or other feature extending between aperture 24 and fixation member 12. Elongated connecting member 22 in the examples of FIGS. 1A, 2A, and 3A defines a circular cross-sectional shape in the Y-Z plane. It should be appreciated, however, that in other examples elongated connecting member 22 may define a different polygonal (e.g., square, triangle, hexagon) or arcuate (e.g., ellipse) shape, and the disclosure is not limited in this respect.

When fixation member 12 and receiving body 14 are assembled as shown in FIG. 1B, anchor body 16 of fixation member 12 is moveably affixed to aperture 24 of receiving body 14 via elongated connecting member 22. In some examples, fixation member 12 and/or elongated connecting member 22 include one or more mechanical attachment features for moveably affixing elongated connecting member 22 to anchor body 16. The mechanical attachment features may help prevent receiving body 14 from detaching from fixation member 12 without user assistance. The mechanical attachment features may also guide the movement of fixation member 12 relative to receiving body 14, e.g., as fixation member 12 translates along elongated connecting member 22 during deployment/fixation.

Different configurations of mechanical attachment features for elongated connecting member are illustrated in each of FIGS. 1A, 2C, and 3C. In the examples of FIGS. 1A and 2C, elongated connecting member 22 includes threading 38 extending along an outer surface of the connecting member. Threading 38 extends along at least a portion of the surface of elongated connecting member 22 from proximal end 34 to distal end 36. Threading 38 may, as illustrated in FIG. 1A, extend along the entire surface from proximal end 34 to distal end 36. Alternatively, threading 38 may, as illustrated in FIG. 2C, extend along less than the entire surface from proximal end 34 to distal end 36 so as to define a threaded portion and a non-threaded portion 37.

Threading 38 may be defined as a continuous or discontinuous ridge (e.g., a helical ridge) extending outwardly (e.g., in the Y-Z plane indicated on FIGS. 1A and 2A) from elongated connecting member 22. In examples where elongated connecting member 22 includes threading 38, fixation member 12 may include complimentary threading extending along at least a portion of interior surface 28 of anchor body 16 (FIG. 1C). For example, fixation member 12 may include complimentary threading extending along the entire length of interior surface 28 of anchor body 16 from proximal end 18 to distal end 20 (FIGS. 1A and 2A), or along a lesser portion of the length of interior surface 28 from proximal end 18 to distal end 20 (FIG. 2F).

In some examples, fixation member 12 includes complimentary threading extending along a lesser portion of the length of interior surface 28 from proximal end 18 to distal end 20 so that threading 38 on receiving body 14 disengages from the complimentary threading extending along an interior surface of fixation member 12 when the receiving body is advanced beyond the location where the complimentary threading ends (e.g., in the negative X-direction indicated on FIG. 2A). An example of such a configuration is illustrated with respect to FIGS. 2E and 2F. As seen in these figures, fixation member 12 in the example of FIGS. 2E and 2F include complimentary threading extending along a portion of the interior surface of fixation member adjacent distal end 20 but not along a portion of the interior surface adjacent proximal end 18. Accordingly, fixation member 12 defines an interior surface with a threaded portion and a non-threaded portion in this example. Threading 38 on elongated connecting member 22 is initially engaged with complimentary threading extending along an interior surface of fixation member 12 (FIG. 2E). As fixation member 12 translates linearly along receiving body 14, the internal threading of fixation member 12 disengages from threading 38 on elongated connecting member 22 as non-threaded portion 37 of elongated connecting member 22 reaches the non-threaded portion of the interior surface of fixation member 12 (FIG. 2F). Upon disengagement, elongated connecting member 22 and fixation member 12 are axially aligned, with threading 38 of elongated connecting member 22 proximally located relative to the internal threading of fixation member 12 (FIG. 2F).

A surgical anchor with a fixation member that is configured to disengage from an elongated connection member as the fixation member is advanced beyond the distal terminus of threading on the elongated connection member may be useful so that the fixation member can move (e.g., rotate) without mechanical impedance from the elongated connecting member. In particular, after fixation member 12 disengages from receiving body 14, a surgeon can continue advancing fixation member 12 into a bone structure without mechanical impedance from elongated connecting member 22 and, corresponding, a surgical attachment element extending through aperture 24.

Independent of the specific length or configuration of threading 38, when threading 38 of elongated connecting member 22 is engaged with the corresponding threading of interior surface 28 of anchor body 16, fixation member 12 is screwably connected to receiving body 14. In examples in which fixation member 12 and receiving body 14 are screwably connected, distal end 20 of fixation member 12 can move relative to aperture 24 of receiving body 14 by rotating fixation member 12 (e.g., clockwise or counter clockwise) relative to elongated connecting member 22. In particular, in the example of FIGS. 1A and 2A, rotating fixation member 12 clockwise relative to a stationary elongated connecting member 22 causes the fixation member to translate towards aperture 24. By contrast, rotating fixation member 12 counter clockwise relative to a stationary elongated connecting member 22 causes the fixation member to translate away from aperture 24. In this manner, the distance separating distal end 20 of fixation member 12 from aperture 24 (e.g., a center of aperture 24) can be controllably varied and, in some examples, set to one of a plurality of different distances, depending on the application.

In different examples, fixation member 12 and/or elongated connecting member 22 may include different mechanical attachment features in addition to or in lieu of threading 38. For instance, in the example of FIG. 3A, the interior surface of anchor body 16 defines at least one radial groove (or radial projection in other examples) extending into the Y-Z plane and elongated connecting member 22 defines at least one corresponding radial projection 41 (or groove in other examples) extending into the same Y-Z plane (FIG. 3C). In this example, fixation member 12 is affixed to receiving body 14 by mating the groove of the interior surface of anchor body 16 with the projection of elongated connecting member 22. When fixation member 12 is advanced toward aperture 24 of elongated connecting member 22, the groove of anchor body 16 disengages from projection 41 of elongated connecting member 22, allowing fixation member 12 to translate linearly along the axis of elongated connecting member 22 (i.e., in the X-direction indicated on FIG. 3A) without rotating either fixation member 12 or receiving body 14. In some examples, fixation member 12 may also include a relief cut 43 (FIG. 3B) so as to resiliently deform during engagement and disengagement of the groove of anchor body 16 and projection 41 of elongated connecting member 22. Surgical anchors with other types of mechanical attachment features are both contemplated and possible, and the disclosure is not limited in this respect.

Surgical anchor 10 includes aperture 24. Aperture 24 is configured to receive surgical attachment element 50. In some examples, aperture 24 receives surgical attachment element 50 by passing one end of the surgical attachment element through the aperture so that one end of the surgical attachment element resides on one side of the aperture and another end of the surgical attachment element resides on another side of the aperture. In other examples, aperture 24 receives surgical attachment element 50 by passing both ends of the surgical attachment element through the aperture so that both ends of the surgical attachment element reside on one side of the aperture and a loop of the surgical attachment element resides on the other side of the aperture.

Aperture 24 can define any suitable cross-sectional shape (e.g., in the X-Z plane indicated on FIGS. 1A, 2A, and 3A). In the examples of FIGS. 1A, 2A, and 3A, aperture 24 defines a trapezoidal cross-sectional shape. However, in other examples, aperture 24 may define a different polygonal (e.g., rectangle, square, triangle, hexagon) or arcuate (e.g., circle, ellipse) shape. Further, in different examples, aperture 24 may define a sharp surface around the periphery of the entrance to the aperture, or aperture 24 may define a rounded surface around the periphery of the entrance to the aperture. In the example of FIG. 2A, aperture 24 defines a rounded surface 23 extending around the periphery of the entrance to the aperture. Rounded surface 23 may ease passage of surgical attachment element 50 through aperture 24. Rounded surface 23 may also prevent damage to surgical attachment element 50 as the attachment element is passed through aperture 24.

In general, surgical attachment element 50 can be any element that is intended to repair tissues during surgery using surgical anchor 10. In one example, surgical attachment element 50 is a tissue approximation device such as a suture. A suture may be constructed from synthetic (e.g., metal, polymer) or biologic (e.g. collagen) materials or any combination thereof, and can be absorbable or nonabsorbable. A suture can further be constructed from monofilament and/or multifilament fibers, assembled in any desired configuration (e.g. braid) to yield any desire properties (e.g., tensile strength). In some examples, a suture may have a diameter ranging from approximately 0.01 mm (size 11-0) to approximately 1.024 mm (size 7). In some additional examples, a suture may be constructed in a tape configuration, such that a width is greater than 1 thickness (e.g., a 5 mm wide polymer tape). Depending on the application, the suture may be attached to a portion of tissue (i.e., either before or after surgical anchor 10 is inserted into a bone structure) to secure the tissue to the bone structure using surgical anchor 10.

In another example, surgical attachment element 50 is a portion of tissue intended to be secured to a bone structure by surgical anchor 10. In these examples, the tissue may be inserted directly into aperture 24, e.g., by passing a portion or end of the tissue through aperture 24, instead of passing an intermediate structure (e.g., suture) through the aperture which in turn is attached to the tissue. In various examples, the tissue may be muscle, a tendon (e.g., tendon stump), a ligament, cartilage, endogenous soft tissue, soft tissue graft (e.g., autograft or allograft), xenograft tissue, cellular scaffolding materials or any other type of tissue. In one specific example, the tissue is a torn tendon stump connected to a bicep muscle. Surgical anchor 10 can be used to secure other types of tissue, however, and the disclosure is not limited in this respect.

In still another example, surgical attachment element 50 is a surgical mesh attachment device, which may also be referred to as a surgical mesh reinforcement device. A surgical mesh attachment device may be wider than a suture to facilitate tissue reconstruction and reinforcement. The increased width of the surgical mesh attachment device may distribute mechanical fixation loads (e.g., tension) over a larger surface area of a tissue repair construct to which the surgical mesh attachment device is applied, which may prevent repair failure or other damage at the tissue site to which the mesh device is attached.

A surgical mesh attachment device can be constructed in a variety of different configurations. Surgical mesh attachment devices can be fabricated from synthetic (e.g. metals, polymers) or biologic (e.g. collagen) materials, or any combination thereof, and can be absorbable or non-absorbable. Surgical mesh attachment devices can further be constructed from monofilament or multifilament fibers, and assemble in a desired configuration (e.g., weave, knit, braid), to yield particular properties (e.g., porosity, tensile strength). Surgical mesh attachment devices derived from biologic materials can be in a graft configuration (e.g., autograft, allograft, xenograft). In some examples, biologic surgical mesh attachment devices can be processed, for example to remove endogenous cells, to attach other biologic agents, to achieve device sterility, or to reconstitute collagen derived from a graft, prior to being used with surgical anchor 10.

In one example, a surgical mesh attachment device includes a mechanical reinforcing component and a cellular scaffold component attached to the mechanical reinforcing component. The mechanical reinforcing component may be formed from a biocompatible material such as, e.g., a biocompatible polymer, metal, or fiber. The cellular scaffold component may be formed of a biocompatible material that facilitates cellular growth and development when located in proximity to living cells such as, e.g., collagen or hydrogel. In some examples, the mechanical reinforcing component has a relatively wide portion and a comparatively narrower end extending from the relatively wide portion, e.g., to facilitate placement and delivery of the device. For example, the mechanical reinforcing component may have a maximum cross-sectional width between approximately 1 mm and approximately 20 mm, and a narrower end that has a width between 0.5 mm and 5 mm. The cellular scaffold component can overlay the entire length or width of the mechanical reinforcing component or a lesser portion of the mechanical reinforcing component (e.g., a wide portion of the component). Depending on the application, the surgical mesh attachment device may be attached to a portion of tissue (i.e., either before or after surgical anchor 10 is inserted into a bone structure) to secure the tissue to the bone structure using surgical anchor 10.

Independent of the specific type of surgical attachment element used for surgical attachment element 50, it should be appreciated that surgical attachment element 50 may be a single attachment element or may include a plurality of surgical attachment elements. For instance, in some examples, surgical attachment element 50 includes a suture arranged in conjunction with other sutures and/or surgical mesh attachment devices. In these examples, the suture(s) and surgical mesh attachment device(s) may both be passed through aperture 24 (e.g., simultaneously) and secured to a bone structure using surgical anchor 10. The ability to utilize a plurality of surgical attachment elements with one anchor device may allow a surgeon to customize a repair construct for a particular injury, without the need for additional and sometimes excessive fixation devices. This ability may also allow the surgeon to achieve a repair construct that includes both mechanical fixation and biological augmentation in a single operation using the same fixation device.

As noted above, aperture 24 can define any suitable cross-sectional size (e.g., in the X-Y plane indicated on FIG. 1B), and the size of aperture 24 may vary. Further, aperture 24 may be configured to receive different types of surgical attachment elements. For this reason, the cross-sectional size of aperture 24 may vary based on the type of surgical attachment element the aperture is configured to receive. In one example, aperture 24 has a cross-sectional area between approximately 0.2 square millimeters (mm²) and approximately 2 mm². Such an example aperture may be configured to receive a single suture strand that has a diameter of approximately 0.5 mm (size 2) and up to 10 strands of the same suture. In another example, aperture 24 has a cross-sectional area between approximately 2 mm² and approximately 20 mm². Such an example aperture may be configured to receive a surgical mesh attachment device that has a cross-sectional area between approximately 2 mm² and approximately 20 mm², and, in some configurations, additional 0.5 mm diameter (size 2) sutures. In still another example, aperture 24 has a cross-sectional area less than or equal to the cross-sectional area of the associated fixation member at its major length. Such an example aperture may be configured to directly receive tissue that has a cross-sectional area approximately equivalent to or less than the cross-sectional area of the associated fixation member at its major length, The foregoing aperture sizes are merely examples, and other aperture sizes are possible.

In some examples, the portion 21 of receiving body 14 defining aperture 24 is sized larger than the cross-sectional area of lumen 32 (FIG. 1C) of anchor body 16. Such an arrangement may prevent the portion 21 of receiving body 14 defining aperture 24 from entering lumen 32 as receiving body 14 is translated proximally toward distal end 20 of anchor body 16. In particular, such an arrangement may prevent surgical attachment element 50 extending through aperture 24 from entering lumen 32 as receiving body 14 is translated proximally toward distal end 20 of anchor body 16. If aperture 24 enters lumen 32 when surgical attachment element 50 extends through the aperture, the portions of the surgical attachment element extending on either side of the aperture may bind against distal end 20 of anchor body 16. Depending on the particular application, such binding may compress and damage (e.g., fray) the surgical attachment element, compromising the repair operation.

Portion 21 of receiving body 14 defines aperture 24. Portion 21 can define any suitable cross-sectional shape (e.g., in the X-Y or X-Z planes indicated on FIGS. 1A, 2A, and 3A). In some examples, portion 21 may includes a proximal to distal taper 40 in the X-Y plane, as illustrated in the example of FIG. 2C, and/or a proximal to distal taper 42 in the X-Z plane as illustrated in FIG. 2D. Taper 40 and/or taper 42 may help facilitate insertion of portion 21 into a pre-formed bone hole or penetration of a bone structure not containing a pre-formed hole.

Aperture 24 can be located at any suitable position on surgical anchor 10. In the example of FIGS. 1A and 1B, aperture 24 is located at the distal end of receiving body 14. In some examples, aperture 24 is transverse to a major axis of fixation member 12. For instance, in the example of FIGS. 1A and 1B, aperture 24 is substantially perpendicular to a major axis extending through anchor body 16 (i.e., in the X-direction indicated on FIG. 1B) when the fixation member 12 is mated with receiving body 14. Other arrangements are possible.

Surgical anchor 10 can be constructed from any of a wide variety of biocompatible materials and can be formed from any material or combination of materials that can provide desired physical, chemical, or biological characteristics. For example, surgical anchor 10 can be fabricated from a polymeric, metallic, or other suitable material. Exemplary materials include, without limitation, polyether ether ketone (PEEK), stainless steel, titanium, polyester, polyoxymethylene (e.g., Delrin®), polysulphones, ultra high molecular weight polyethylene (UHMWPE), absorbable polymers (e.g., polylactic acid, polyglycolic acid, and so forth), reinforced polymers (e.g., fiber reinforced polymer matrices), polymer blends, copolymers, composite materials, bone (e.g., artificial bone, cadaver bone, or the like) and combinations thereof.

Surgical anchor 10 can be formed using any acceptable technique including, without limitation, machining, extrusion, molding, fused deposition modeling, selective laser sintering, stereolithography, and the like. For example, a polymeric or metal fixation devices can be formed through multi-axis machining, according to methods generally known to those of ordinary skill in the art. Other machining methods that may be utilized include, without limitation, lathing, milling, electrical discharge machining (EDM), stamping, and the like. By way of example, extrusion methods can include multi-phase as well as step extrusion methods, as are generally known. Molding methods as may be utilized can include injection molding, pulltrusion molding, rotomolding, solvent molding, cast molding, compression molding, polymerization molding (i.e., monomers and/or oligomers are polymerized within the mold), and so forth. Of course, multiple formation methods can be utilized in conjunction with one another as well.

Surgical anchor 10 (with or without an associated inserter device) can be provided as a sterile or non-sterile device, depending upon the desired application. When considering sterile devices, any sterilization procedures can be utilized as is generally known in the art. For example, a device can be sterilized by liquid chemical, gas chemical, radiation, or any other sterilization process.

While surgical anchor 10 in the examples of FIGS. 1A, 2A, and 3A is described above as including two separate, connectible features—fixation member 12 and receiving body 14—it should be appreciated that the disclosure is not limited to such an example configuration of features. In different examples, surgical anchor 10 may define a unitary structure (e.g., where receiving body 14 is not separable from fixation member 12) or may include three, four, or more separate, connectible features.

FIG. 4 is a schematic drawing of an example anchor inserter 100 that can be used to insert surgical anchor 10 into a bone structure. During surgery, surgical anchor 10 is detachably connected to anchor inserter 100 to deliver the anchor to a surgical fixation site, e.g., during arthroscopic surgery. Thereafter, anchor inserter 100 is used to insert the anchor into a bone hole, e.g., by engaging threading 26 of fixation member 12 with a sidewall of the bone hole, to secure the anchor in the bone hole.

Anchor inserter 100 can assume a variety of different configurations. However, in the example of FIG. 4, anchor inserter 100 includes an outer driver 102 and inner rod 104 (which are shown separated in FIG. 4). Outer driver 102 includes a shaft that defines a lumen extending from proximal end 106 to a distal end 108. Distal end 108 of outer driver 102 is configured to connect to proximal end 18 of fixation member 12. Inner rod 104 includes a rod extending from proximal end 110 to distal end 112. Distal end 112 of inner rod 104 is configured to connect to receiving body 14.

Anchor inserter 100 is assembled by connecting distal end 108 of outer driver 102 to fixation member 12 and inserting inner rod 104 through the lumen defined by outer driver 102. In such an arrangement, inner rod 104 and outer driver 102 are coaxially aligned. In addition, in examples where fixation member 12 defines a lumen extending through anchor body 16 (e.g., FIG. 1A), inner rod 104 may also extend partially or fully through the lumen defined by the anchor body. Distal end 112 of inner rod 104 is connected to receiving body 14 and, in some examples, may extend partially or fully through elongated connecting member 22. When so assembled, outer driver 102 is mated with fixation member 12 and inner rod 104 is mated with receiving body 14.

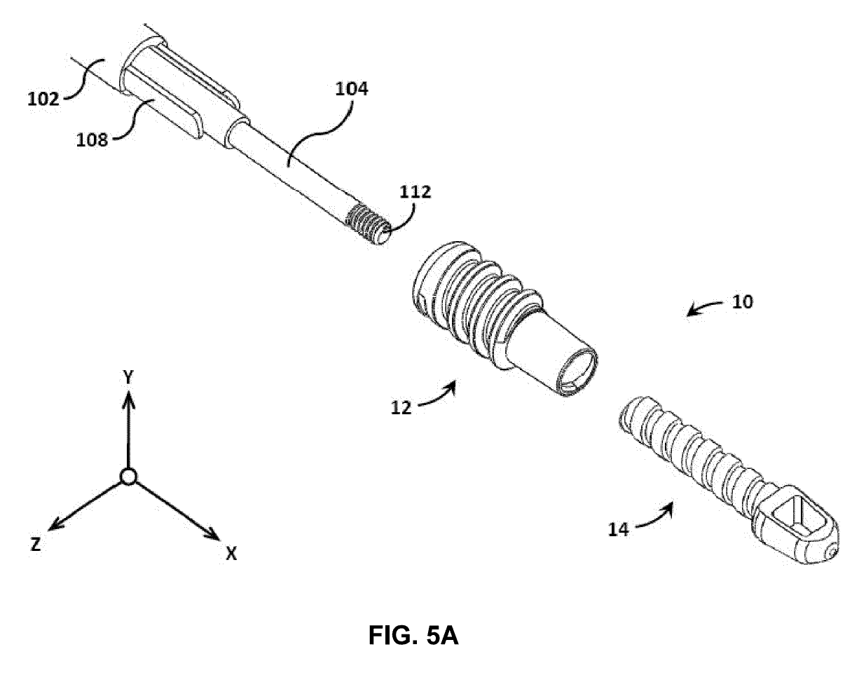

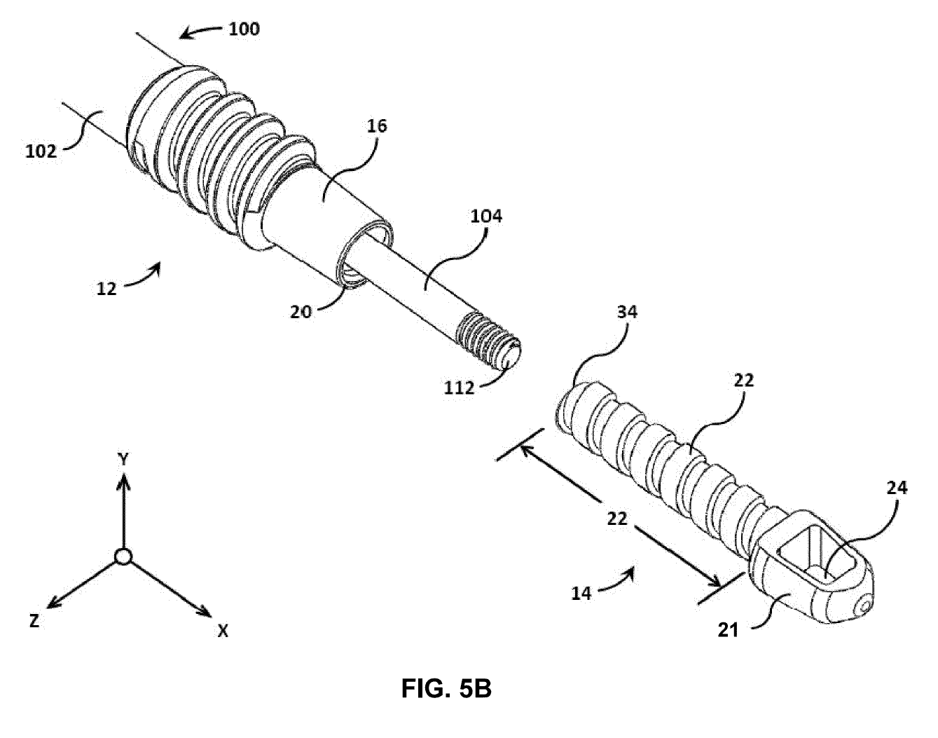

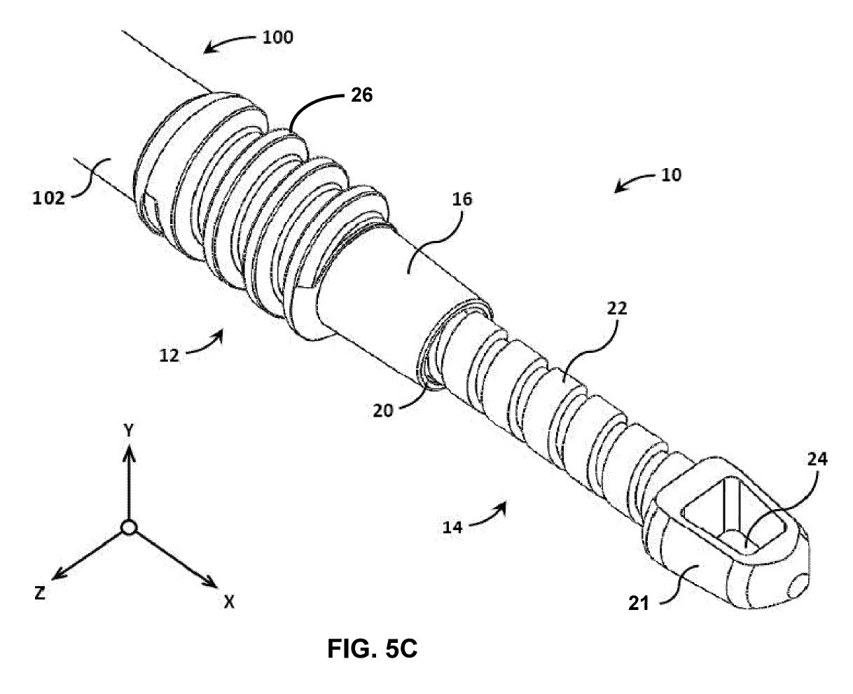

FIGS. 5A-5C are enlarged views of the distal end of anchor inserter 100 connected to surgical anchor 10. FIG. 5A illustrates an enlarged view of surgical anchor 10 and anchor inserter 100 prior to attachment. FIG. 5B illustrates outer driver 102 of anchor inserter 100 connected to fixation member 12. Inner rod 104 extends through a lumen defined by outer driver 102 and fixation member 12 in this example. FIG. 5C illustrates inner rod 104 subsequently connected to receiving body 14 and fixation member 12 mated with receiving body 14 to define an assembled surgical anchor 10. In this manner, anchor inserter 100 is connected to surgical anchor 10 to define an assembled system that includes an anchor inserter and a surgical anchor.

Anchor inserter 100 is configured to secure surgical anchor 10 to a bone structure. Accordingly, the specific design of anchor inserter 100 may vary based, e.g., on the specific design of surgical anchor 10. In the example of FIG. 5C, surgical anchor 10 includes threading 26 extending around an external surface of anchor body 16. Threading 26 is configured to mechanically engage with a sidewall of a bone hole to secure surgical anchor 10, and a surgical attachment element extending through aperture 24 of surgical anchor 10, in the bone hole. In such an example, a surgeon can position receiving body 14 of surgical anchor 10 (while connected to anchor inserter 100) in a bone hole and rotate outer driver 102 to insert the anchor into the bone hole. In alternative examples where fixation member 12 includes plug features, the surgeon can position receiving body 14 of surgical anchor 10 (while connected to anchor inserter 100) in a bone hole and impact the fixation member into the bone hole. In these examples, the surgeon may apply a force substantially axial with the direction of the bone hole to forcibly insert the surgical anchor into the bone hole. Alternative applications and configurations of anchor inserter 100 are contemplated.

In general, outer driver 102 of anchor inserter 100 is configured to move independently of inner rod 104 of anchor inserter 100. Depending on the configuration of surgical anchor 10, independent movement of outer driver 102 relative to inner rod 104 can independently move fixation member 12 relative receiving body 14. For instance, in the example of FIG. 5C, surgical anchor 10 defines a rotatable connection between fixation member 12 and receiving body 14. Accordingly, in this example, rotating outer driver 102 (connected to fixation member 12) relative to inner rod 104 (connected to receiving body 14) results in the rotation of fixation member 12 relative to receiving body 14. In turn, this relative movement causes fixation member 12 to translate (e.g., either proximally or distally) relative to aperture 24 of receiving body 14.

The ability to independently position fixation member 12 relative to aperture 24 may be useful in a variety of different surgical situations. For example, when using surgical anchor 10 to secure surgical attachment element 50 (FIG. 1B) to a bone structure, a surgeon can place the surgical attachment element through aperture 24 of receiving body 14 and position aperture 24 into a bone hole such that a desired tension is placed upon the surgical attachment element. The surgeon can subsequently rotationally insert fixation member 12 into the bone structure, thereby mechanically engaging the bone hole and securing the surgical attachment element.

Depending on the configuration of surgical anchor 10, translation of fixation member 12 relative to aperture 24 may change the overall length of surgical anchor 10, allowing the surgeon to adjust the length until the length corresponds to, or is smaller than, the depth of the bone hole in which the anchor is to be inserted. If the tension placed on surgical attachment element 50 is not satisfactory to the surgeon (i.e. greater or lesser than desired), the surgeon may reversibly rotate outer driver 102 relative to inner rod 104 to reversibly translate fixation member 12 relative to aperture 24. In this way, the surgeon can mechanically disengage surgical anchor 10 from the bone hole, thereby releasing surgical attachment element 50 from fixation. Once surgical attachment element 50 is released from fixation, the surgeon can adjust the amount of tension placed on the surgical attachment element, e.g., by pulling the surgical attachment one direction or the other through aperture 24, before reestablishing fixation.

In some configurations, a surgeon may use anchor inserter 100 to adjust the tensioning and/or positioning of surgical attachment element 50 after surgical anchor 10 is secured in a bone hole. For example, as described above with respect to FIGS. 2A, 2E, and 2F, threading 38 may extend a specified distance along elongated connecting member 22 of receiving body 14, such that fixation member 12 can become disengaged from receiving body 14 at a location corresponding to the distance of threading 38 along elongated connecting member 22 (e.g., as the distance between the distal end of fixation member 12 and the distal end of receiving body 14 is decreased). Such a configuration may allow the surgeon to further advance the fixation member even after it has translated the entire length of the elongated connecting member of the receiving body and/or the distal end 20 of the fixation member contacts distal portion 21 of the receiving body. If, after securing surgical anchor 10 in the bone hole, the surgeon decides that the tension on, or position of, the surgical attachment element is not appropriate, the surgeon can increase-tension on the surgical attachment element without extracting the surgical anchor from the bone hole. Further advancement of the fixation member into the bone hole will advance aperture 24, and surgical attachment element 50, deeper into the bone hole, thereby increasing the amount of tension placed on surgical attachment element 50.

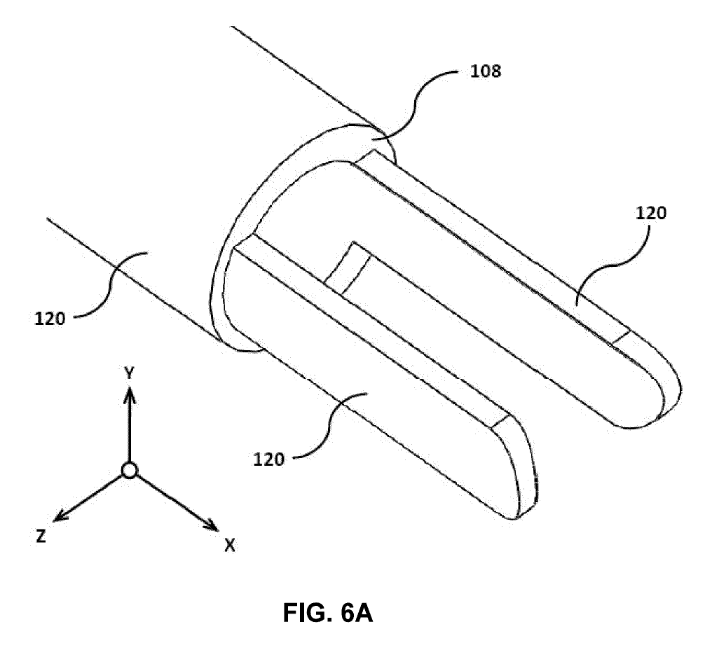

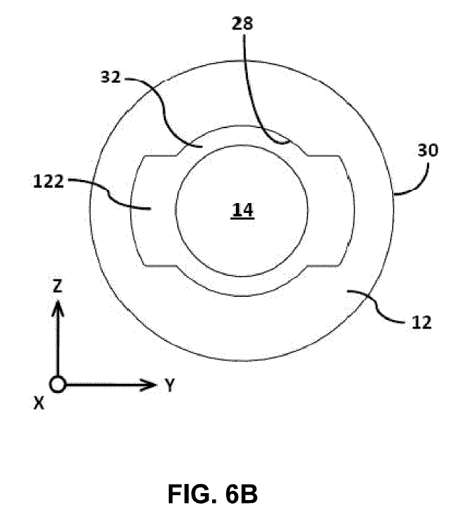

As briefly described above, outer driver 102 of anchor inserter 100 is configured to detachably connect to fixation member 12 of surgical anchor 10. Outer driver 102 can include any features suitable for mechanically attaching the driver to the fixation member. FIGS. 6A and 6B illustrate one example of corresponding connection features that can be used to connect outer driver 102 to fixation member 12.

As seen in FIG. 6A, outer driver 102 includes at least one connecting prong 120 extending from distal end 108 of the outer driver which, in the example of FIG. 6A, is illustrated as two connecting prongs. Connecting prong 120 extends parallel to the major axis of outer driver 102 and is configured (e.g., sized and shaped) to mate with a corresponding channel on fixation member 12. Connecting prong 120 can extend any suitable distance along the length of the major axis of fixation member 12, and increasing the length of connecting prong 120 may increase the distribution of force over fixation member 12 during insertion. Increasing force distribution over fixation member 12 during insertion may help prevent torsion failure.

FIG. 6B illustrates an example corresponding connection channel on fixation member 12. In particular, FIG. 6B illustrates an example of proximal end 18 of fixation member 12 which includes at least one channel 122 (illustrated as two channels) that is configured (e.g., sized and shaped) to receive connecting prong 120 of outer driver 102. Channel 122 is interposed between lumen 32 and exterior surface 30 of anchor body 16.

Connecting prong 120 is connected to channel 122 by inserting the prong of outer driver 102 into the channel of fixation member 12 (i.e., in the negative X-direction indicated on FIG. 6B). During use, connecting prong 120 transfers force (e.g., torque) from the handle of outer driver 102 into fixation member 12 to secure the fixation member in a bone hole. Specifically, with the example configuration of fixation member 12 of FIGS. 1A and 2B, connecting prong 120 transfers torque from the handle of outer driver 102 to fixation member 12 to impart a rotational motion to the fixation member, thereby advancing the fixation member into the bone hole.

It should be appreciated that the corresponding connection features in FIGS. 6A and 6B are merely examples, and alternative connection features are both possible and contemplated. For instance, in different examples, outer driver 102 and fixation member 12 may include fewer connection prongs and corresponding connection channels (e.g., a single prong and channel) or more connection prongs and corresponding connection channels (e.g., three, four, or more prongs and channels) than illustrated in FIGS. 6A and 6B. In addition, in further examples, the position of connecting prong 120 and channel 122 may be reversed so that fixation member 12 includes the connection prong(s) and outer driver 102 includes the channel(s). In another example, corresponding connection channels may be defined on an exterior surface of fixation member 12 (e.g., a recessed area of fixation member 12) rather than as internal channels.

In still further examples, fixation member 12 and outer driver 102 may not be configured to connect via a prong and channel arrangement but may instead include different attachment features such as, e.g., threaded connectors, magnetic connectors, or the like. In one example, one of fixation member 12 and outer driver 102 may include a polygonal (e.g., hexagonal) shaped male connector, and the other of the fixation member 12 and outer driver 102 may include female connector sized and shaped to receive the male connector.

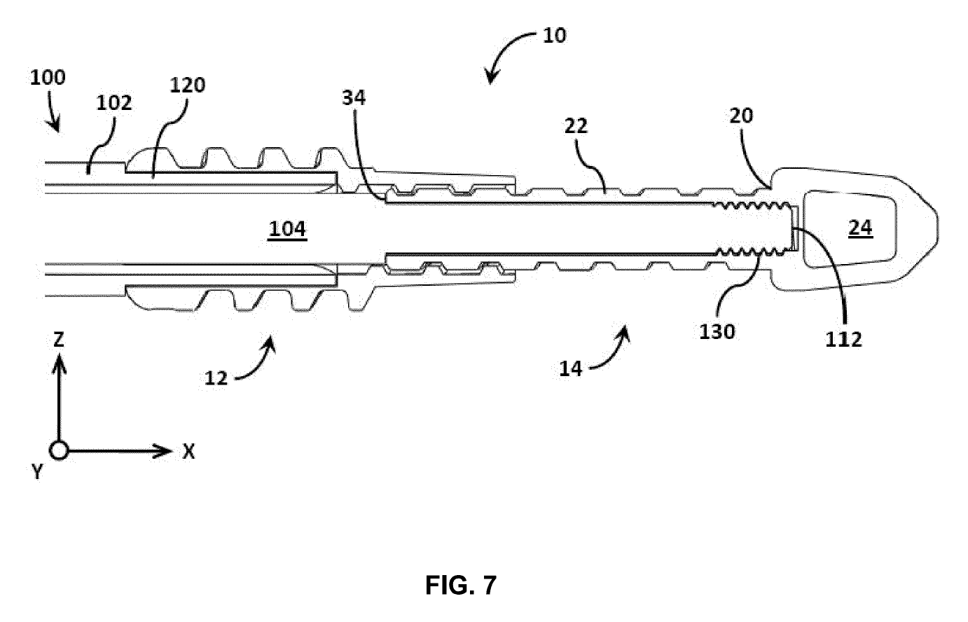

Inner rod 104 of anchor inserter 100 is configured to detachably connect to receiving body 14 of surgical anchor 10. As with outer driver 102, inner rod 104 can include any features suitable for mechanically attaching the rod to the receiving body. FIG. 7 is a cross-sectional schematic illustrating anchor inserter 100 connected to surgical anchor 10 of FIGS. 1A-1C. FIG. 7 illustrates one example of corresponding connection features that can be used to connect inner rod 104 to receiving body 14.

In the example of FIG. 7, inner rod 104 includes a threaded connector 130 at distal end 112 of the rod. Threaded connector 130 is configured to connect to a corresponding threaded connector on receiving body 14 that extends partially or fully along elongated connecting member 22. In some examples, the corresponding threaded connector on receiving body 14 is located at proximal end 34 of elongated connecting member 22. In other examples, as illustrated in FIG. 7, the corresponding threaded connector on elongated connecting member 22 is located at distal end 36 of the elongated connecting member. When the corresponding threaded connector on receiving body 14 is located at distal end 36 of the elongated connecting member, the elongated connecting member may define a lumen extending at least partially, and in some case fully, through the major axis of the elongated connecting member. The lumen is configured to receive inner rod 104 of anchor inserter 100.

While the corresponding threaded connector on elongated connecting member 22 may be located at any position along elongated connecting member 22, positioning the threaded connector at distal end 36 of the elongated connecting member may transfer force to the region adjacent aperture 24. This may prevent inner rod 104 from inadvertently disengaging (e.g., breaking away) from receiving body 14 when moving the receiving body. Further, inner rod 104 may function as a reinforcing member when the rod extends through elongated connecting member 22. The corresponding connection features in FIG. 7 are merely examples, and alternative connection features are possible.

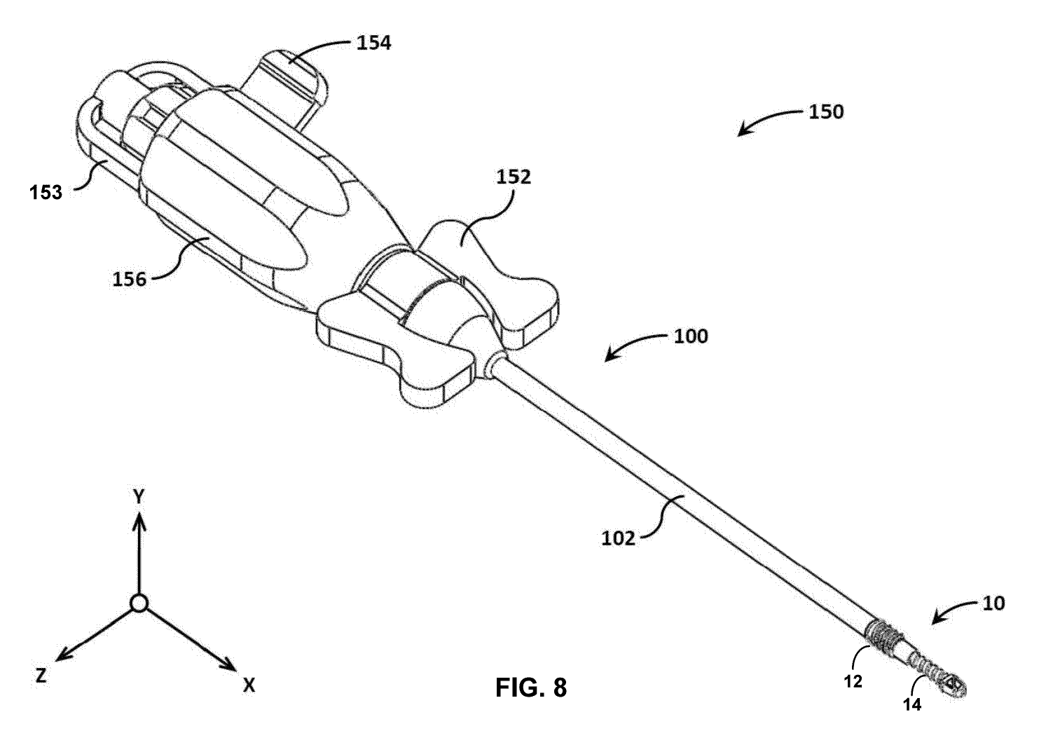

FIG. 8 is a schematic illustration of an example system 150 that includes surgical anchor 10 and anchor inserter 100. As illustrated, system 150 includes previously-described outer driver 102, inner rod 104 (not visible in figure), fixation member 12 and receiving body 14. System 150 also includes optional swivel grip 152 and guard 153. Guard 153 helps prevent inadvertent contact of inner rod 104 during movement of outer driver 102. Guard 153 may also provide a location for impacting to impart an axial force to surgical anchor 10. Swivel grip 152 is configured to connect and rotate around handle 156 of outer driver 102. Swivel grip 152 may provide a stationary gripping location for a surgeon to grip anchor inserter 100 while inserting surgical anchor 10. Swivel grip 152 may also provide axial stability to surgical anchor 10, e.g., to help maintain axial alignment of the anchor with a bone hole during insertion.

In some examples, anchor inserter 100 also includes a locking pin 154 extending transversely through outer driver 102 and inner rod 104. Locking pin 154 may prevent outer driver 102 from moving (e.g., rotating) relative to inner rod 104, and therefore movement between fixation member 12 and receiving body 14, until the pin is removed. Depending on the application, the pin may be used relieve stress from the mating interface of fixation member 12 and receiving body 14 during insertion of aperture 24 into a bone hole, or to maintain a specific configuration of surgical anchor 10 during transport and storage of anchor inserter 100.

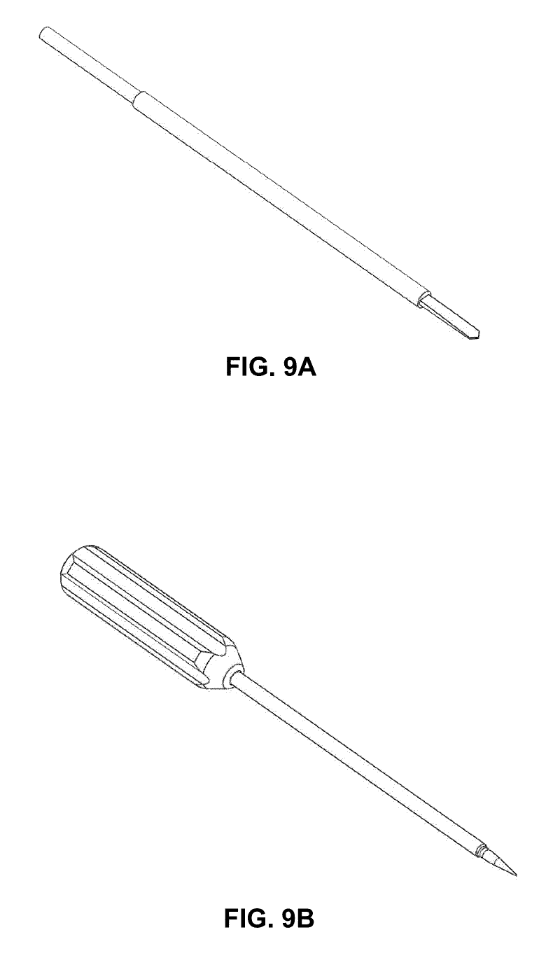

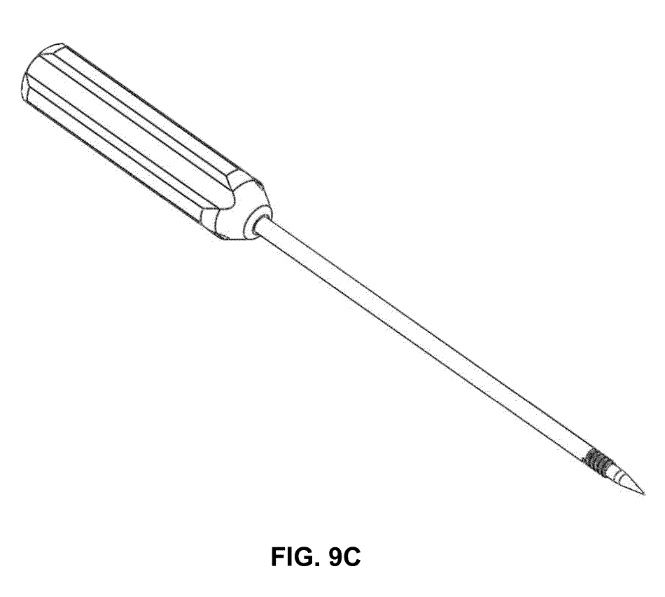

FIGS. 9A-9C are conceptual illustrations of example surgical tools that may, but need not, be used when surgically implanting surgical anchor 10 into a bone structure. One or more (e.g., all) of the surgical tools may be included in a kit of parts that includes surgical anchor 10 and/or anchor inserter 100 and/or a surgical attachment element (e.g., surgical attachment element 50).

FIG. 9A is a conceptual illustration of an example spade drill that may be used to form a hole in a bone structure. FIG. 9B is an example punch that may be used to form a hole in a bone structure in lieu of the spade drill of FIG. 9A. FIG. 9C is an example tap that may be used to form a hole in a bone structure in addition to, or in lieu of, the spade drill of FIG. 9A or the punch of 9B.

FIGS. 10-21 conceptually illustrate example steps that may be performed during an example technique for securing a surgical attachment element to a bone structure using a surgical anchor. For ease of description, the technique of FIGS. 10-21 is described with respect to surgical anchor 10 (FIGS. 1, 2, and 3) and anchor inserter 100 (FIGS. 4-8). A similar technique can be used with surgical anchors and/or anchor inserters having configurations other than those described above with respect to FIGS. 1-8.

With reference to FIGS. 10 and 11, the example technique of FIGS. 10-21 involves securing a portion of tissue 200 to a bone structure 202 using surgical attachment element 50. Surgical attachment element 50 is an element that is intended to be attached to bone structure 202 during surgery using surgical anchor 10. In the example of FIG. 10, surgical attachment element 50 is illustrated as a fiber suture that has a cross-sectional diameter of approximately 0.05 mm (size 2). In the example of FIG. 11, surgical attachment element 50 is illustrated as a surgical mesh attachment device that includes a cellular scaffold component 204 with mechanical reinforcing component 206 extending from opposed ends of cellular scaffold component 204. Cellular scaffold component 204 has a major cross-sectional width of approximately 7 mm in the example of FIG. 11. Additional or different surgical attachment elements may be used, as described above.

In FIGS. 10 and 11, surgical attachment element 50 is attached to tissue 200 (e.g., passed through or threaded through tissue 200) before the surgical attachment element is secured to bone structure 202 using surgical anchor 10. This allows tissue 200 to be secured to bone structure 202 via surgical attachment element 50. In different examples, surgical attachment element 50 is secured to bone structure 202 before attaching the surgical attachment element through tissue 200. In these examples, surgical attachment element 50 is attached to tissue 200 after the surgical attachment element is secured to bone structure 202 using surgical anchor 10. Any suitable tissue passing device may be used to attach surgical attachment element 50 to tissue 200 including, for example, a free needle, a tissue weaving forceps, or an arthroscopic suture shuttle device. Further, any acceptable technique may be used to secure surgical attachment element 50 to soft tissue including, e.g., knot tying, weaving through tissue, or secondary knotless anchor fixation.

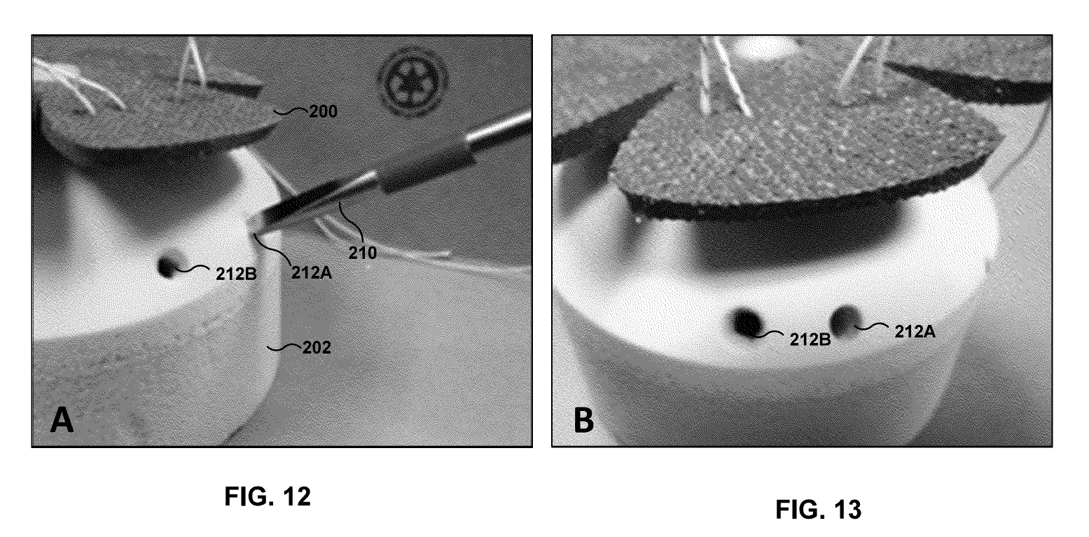

Surgical anchor 10 is configured to be inserted into a bone hole formed in bone structure 202. Accordingly, during an example attachment technique, a surgeon may form a pilot bone hole in bone structure 202 to guide surgical anchor 10 into the bone structure. FIG. 11 illustrates drill 210 forming at least one bone hole 212 in bone structure 202, which in the example of FIG. 11 is illustrated as two bone holes 212A and 212B (collectively “bone hole 212”). The completed bone holes are illustrated in FIG. 12. In different examples, the surgeon may punch, tap, or use any other acceptable technique to form bone hole 212 in addition to or in lieu of drilling.

Independent of the specific technique used to form bone hole 212, the surgeon may form the bone hole so that the bone hole exhibits a cross-sectional width that is smaller than the cross-sectional width of fixation member 12 (e.g., in the Y-direction indicated in FIG. 1A). When the cross-sectional width of bone hole 212 is less than the cross-sectional width of fixation member 12, fixation member 12 can mechanically engage with the sidewall of bone hole 212 when the fixation member is inserted into the bone hole. This mechanical engagement can secure (e.g., fixate) fixation member 12 in bone hole 212.

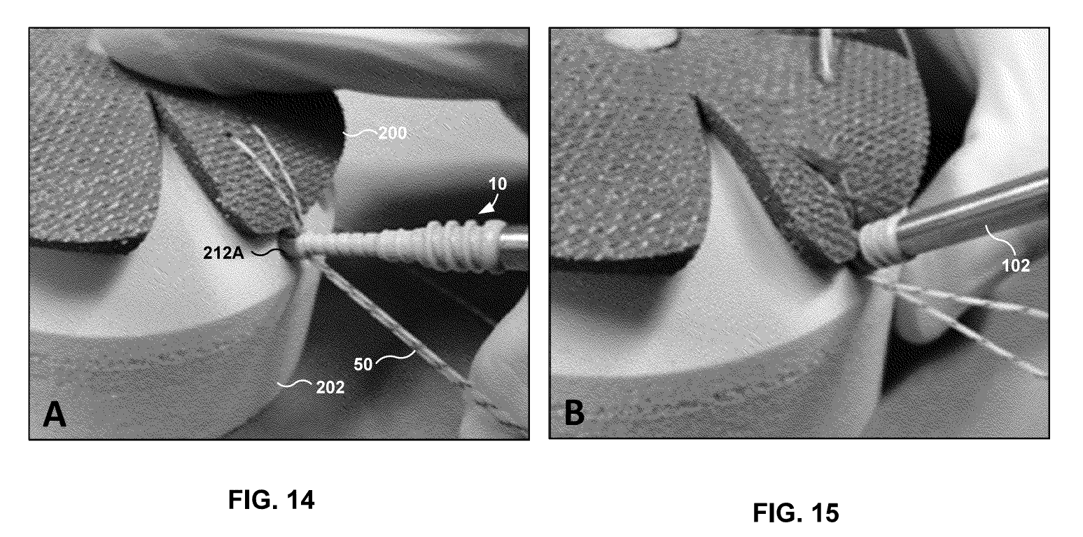

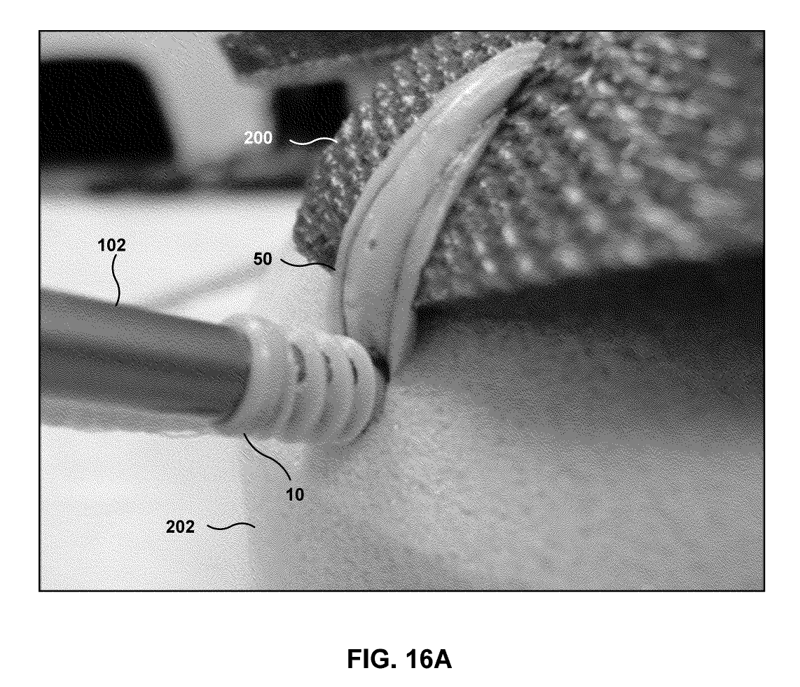

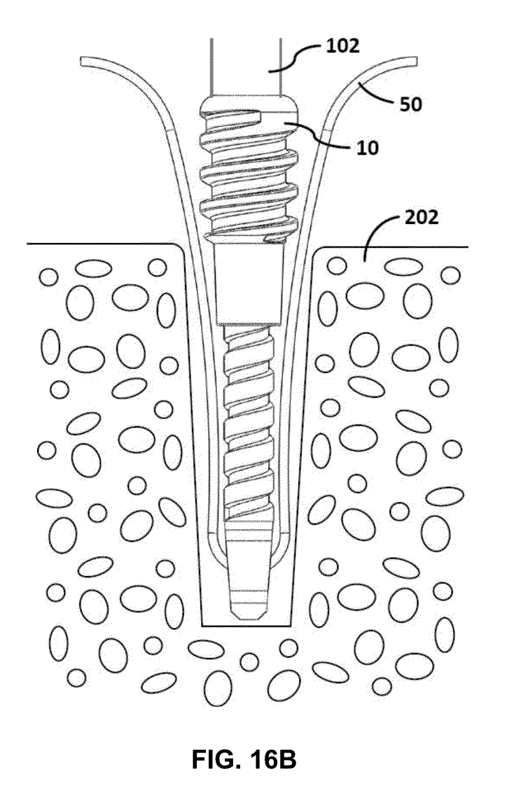

In addition to forming bone hole 212, the example technique of FIGS. 10-21 involves passing surgical attachment element 50 through aperture 24. In some examples, as illustrated in FIG. 14, two free ends of surgical attachment element 50 are passed through aperture 24 so that a loop of the surgical attachment element (e.g., a loop passing through tissue 200) resides on one side of the aperture and two free ends of the surgical attachment element reside on the other side of the aperture. In other examples, as illustrated in FIG. 16, a single free end of surgical attachment element 50 is passed through aperture 24 so that one end of the surgical attachment element resides on one side of the aperture and another end of the surgical attachment element resides on another side of the aperture.

With surgical attachment element 50 attached to tissue 200 and passed through aperture 24, the surgeon may adjust the tension on and/or position of tissue 200. In various examples, the surgeon may pull on surgical attachment element 50 and/or physically move tissue 200 relative to bone hole 212 to adjust the tension and/or position tissue 200 relative to bone hole 212. Properly tensioning and positioning tissue 200 relative to bone structure 202 can help ensure that the tissue is subsequently secured to bone structure 202 in a manner that promotes healing and recovery.

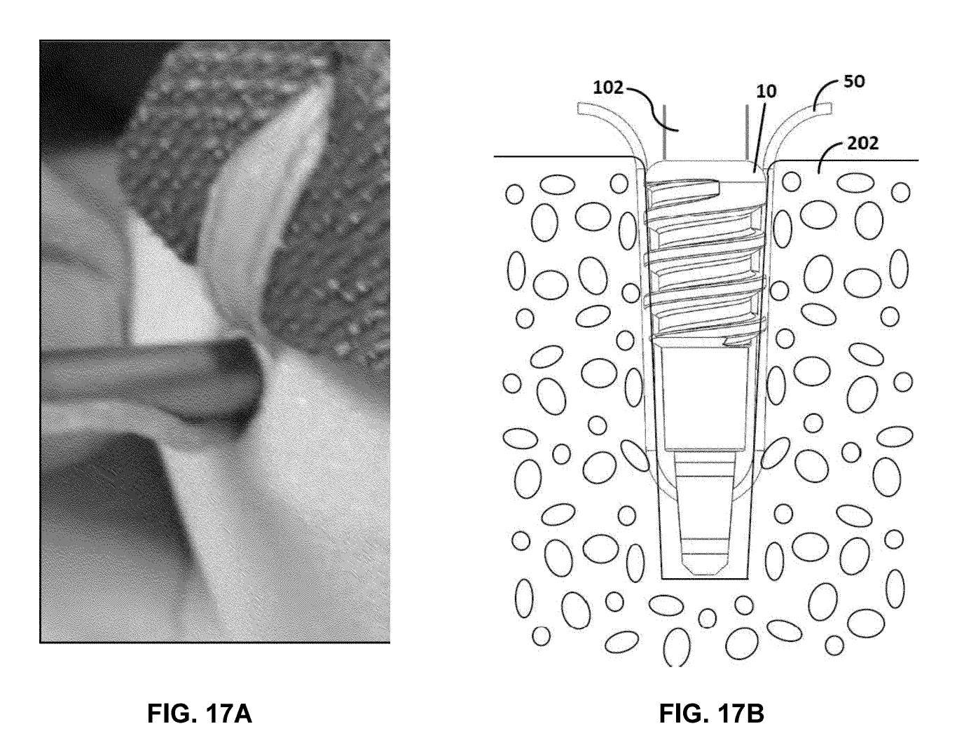

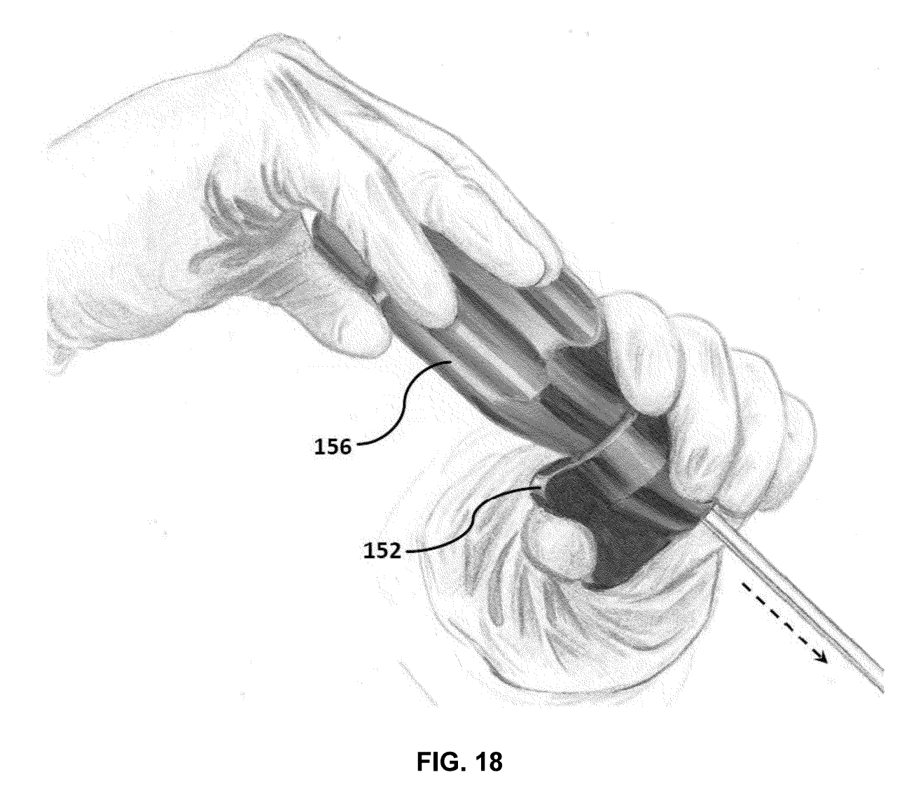

After adjusting the tension on and/or position of tissue 200, the surgeon can insert surgical anchor 10 into bone hole 212 to secure the tissue to bone structure 202. Specifically, the surgeon can insert portion 21 of surgical anchor 10 defining aperture 24 into bone hole 212 and thereafter advance fixation member 12 into the bone hole to secure the anchor in the bone hole. This is conceptually illustrated in FIGS. 14-18. As shown in FIGS. 14, 15, 16A, and 16B, the surgeon may initially insert portion 21 of elongated connecting member 22 defining aperture 24 into bone structure 202. The surgeon may then rotate outer driver 102 clockwise (or counter clockwise in different examples) as illustrated in FIG. 18, causing fixation member 12 to rotate clockwise and translates distally into bone hole 212. Fixation member 12 rotates around threading 38 of elongated connecting member 22 (FIG. 1A) and translates along elongated connecting member 22 (e.g., in the X-direction indicated on FIG. 1A). While fixation member 12 moves relative to receiving body 14, threading 26 on the external surface of anchor body 16 (FIG. 1A) engages with the sidewall of bone hole 212. In some examples, elongated connecting member 22 is held substantially stationary while fixation member 12 rotates about elongated connecting member 22. For instance, in one example, elongated connecting member 22 is held substantially stationary by the force of surgical attachment element 50 extending through aperture 24 and out of bone hole 212. In another example, the surgeon holds inner rod 104 while rotating outer driver 102 about inner rod 104 to hold elongated connecting member 22 substantially stationary. In either set of examples, the surgeon may insert surgical anchor 10 into bone hole 212 until proximal end 18 of fixation member 12 is substantially flush with bone structure 202, as illustrated in FIGS. 17A and 17B. Alternatively, the surgeon may insert fixation member 12 to a countersink depth, e.g., to avoid irritation of surrounding tissue.

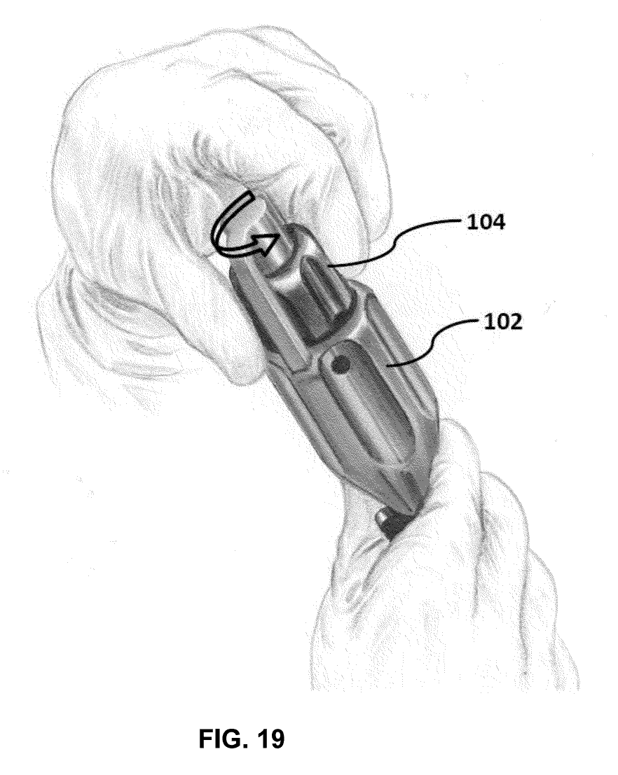

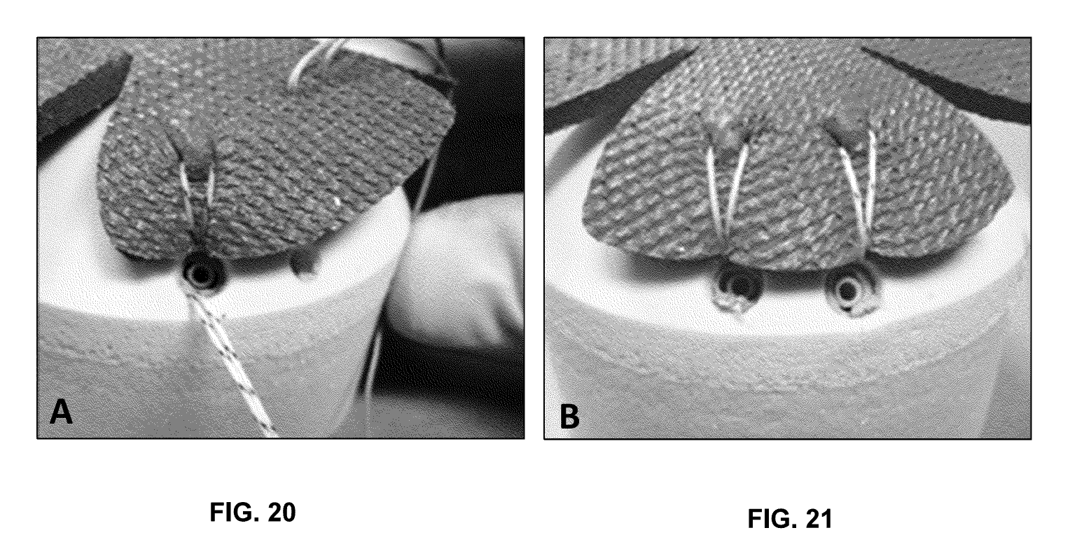

After suitably adjusting the tension and/or position of tissue 200 and securing anchor 10 into the bone structure, the method of FIGS. 10-21 include disengaging anchor inserter 100 from surgical anchor 10. Anchor inserter 100 is disengaged from surgical anchor 10 by rotating inner rod 104 counter clockwise as shown in FIG. 19 to disconnect threaded connector 130 (FIG. 7) from the corresponding threaded connector on receiving body 14. During this disengagement, aperture 24 may be held stationary by surgical attachment element 50 as the surgical attachment element is tensioned across aperture 24 and fixed within bone structure 202. Thereafter, outer driver 102 is pulled axially away from fixation member 12 (e.g., in the negative X-direction indicated on FIG. 1A) to release connecting prong 120 (FIG. 6A) from channel 122 (FIG. 6B) of fixation member 12. In this manner, anchor inserter 100 is released from surgical anchor 10 to leave a secured anchor as illustrated in FIG. 20.

Alternatively, if the tension placed on surgical attachment element 50 is not satisfactory to the surgeon (e.g., greater or lesser than desired), the surgeon may reversibly rotate outer driver 102 relative to inner rod 104 to reversibly translate fixation member 12 relative to aperture 24. Reversibly rotating fixation member 12 mechanically disengages surgical anchor 10 from bone hole 212, thereby releasing surgical attachment element 50 from fixation. With surgical attachment element 50 released from fixation, the surgeon can adjust the positioning and/or amount of tension placed on the surgical attachment element before reestablishing fixation. Excess surgical attachment element may be trimmed away as shown in FIG. 20

Various examples have been described. These and other examples are within the scope of the following claims.

1. A surgical anchor comprising:
a fixation member that includes an anchor body extending between a proximal end and a distal end; and
a receiving body that includes an elongated connecting member extending between a proximal end and a distal end, the receiving body including an aperture defined at the distal end of the elongated connecting member that is configured to receive a surgical attachment element,
wherein the receiving body is connected to the fixation member via the elongated connecting member, and the fixation member is configured to move relative to the receiving body.

2. The surgical anchor of claim 1, wherein the fixation member is a fixation screw that includes exterior helical threading extending along at least a portion of an exterior surface of the anchor body.

3. The surgical anchor of claim 1, wherein the fixation member is a fixation plug that includes exterior ribbing extending along at least a portion of an exterior surface of the anchor body.

4. The surgical anchor of claim 1, wherein the fixation member includes connecting threading, the elongated connecting member of the receiving body includes complimentary threading that is configured to mate with the connecting threading of the fixation member, and the fixation member is configured to move relative to the receiving body by rotating the fixation member relative to the receiving body.

5. The surgical anchor of claim 4, wherein the connecting threading of the fixation member extends less than an entire length of the fixation member, and the complimentary threading of the elongated connecting member extends less than an entire length of the elongated connecting member so that the connecting threading of the fixation member disengages from the complimentary threading of the elongated connecting member as the distal end of the fixation member advances towards the distal end of the receiving body.

6. The surgical anchor of claim 4, wherein the anchor body defines a lumen extending from a proximal end of the anchor body to a distal end of the anchor body, the anchor body defines an interior surface facing the lumen and an exterior surface opposite the interior surface, and the connecting threading extends along at least a portion of the interior surface of the anchor body, and wherein the proximal end of the elongated connecting member of the receiving body is inserted into the distal end of the anchor body with the connecting threading of the anchor body mated with the complimentary threading of the elongated connecting member.

7. The surgical anchor of claim 6, wherein the elongated connecting member is an elongated pin that defines a major length and a cross-sectional width, and the major length is greater than the cross-sectional width.

8. The surgical anchor of claim 6, wherein a major length of the elongated connecting member is at least as long as a major length of the lumen of the fixation member.

9. The surgical anchor of claim 6, wherein a portion of the receiving body defining the aperture is larger than a cross-sectional width of the lumen so as to prevent the receiving body from being inserted into the lumen of the fixation member.

10. The surgical anchor of claim 6, wherein the anchor body defines at least two channels interposed between the lumen and the exterior surface of the anchor body, the at least two channels opening towards the proximal end of the anchor body, and the at least two channels configured to receive at least two connection prongs of an anchor inserter.

11. The surgical anchor of claim 1, wherein the fixation member includes a connecting groove or rib, the elongated connecting member of the receiving body includes a complimentary rib or groove that is configured to mate with the connecting groove or rib of the fixation member, and the fixation member is configured to move relative to the receiving body without rotating the fixation member relative to the receiving body.

12. The surgical anchor of claim 1, wherein the aperture is transverse to a major axis of the fixation member.

13. The surgical anchor of claim 1, wherein the aperture is configured to receive a surgical attachment element that has a cross-sectional area greater than 20 square millimeters.

14. The surgical anchor of claim 1, wherein the fixation member further comprises at least one aperture extending generally transverse to a major axis of the anchor body to facilitate bone in growth upon implantation.

15. The surgical anchor of claim 1, wherein the surgical anchor comprises a biocompatible material.

16. The surgical anchor of claim 15, wherein the surgical anchor comprises titanium.

17. The surgical anchor of claim 15, wherein the surgical anchor comprises poly ether ether ketone (PEEK).

18. A surgical anchor system comprising:
(A) an anchor inserter that includes:
(i) a shaft defining a lumen extending from a proximal end of the shaft to a distal end of the shaft; and
(ii) a rod extending through the lumen defined by the shaft; and
(B) a surgical anchor that includes:
(i) a fixation member that includes an anchor body extending between a proximal end and a distal end; and
(ii) a receiving body that includes an elongated connecting member extending from a proximal end to a distal end, the receiving body including an aperture defined at the distal end of the elongated connecting member that is configured to receive a surgical attachment element,
wherein the fixation member is connected at the distal end of the shaft, the receiving body is connected a distal end of the rod, and the receiving body is connected to the fixation member via the elongated connecting member so that moving the shaft relative to the rod moves the fixation member relative to the receiving body.

19. The surgical anchor system of claim 18, wherein the fixation member is a fixation screw that includes exterior helical threading extending along at least a portion of an exterior surface of the anchor body.

20. The surgical anchor system of claim 18, wherein the fixation member is a fixation plug that includes exterior ribbing extending along at least a portion of an exterior surface of the anchor body.

21. The surgical anchor system of claim 18, wherein the fixation member includes connecting threading, the elongated connecting member of the receiving body includes complimentary threading that is configured to mate with the connecting threading of the fixation member, and the fixation member is configured to move relative to the receiving body by rotating the shaft relative to the rod.

22. The surgical anchor system of claim 21, wherein the connecting threading of the fixation member extends less than an entire length of the fixation member, and the complimentary threading of the elongated connecting member extends less than an entire length of the elongated connecting member so that the connecting threading of the fixation member disengages from the complimentary threading of the elongated connecting member as the distal end of the fixation member advances towards the distal end of the receiving body.

23. The surgical anchor system of claim 21, wherein the anchor body defines a lumen extending from a proximal end of the anchor body to a distal end of the anchor body, the anchor body defines an interior surface facing the lumen and an exterior surface opposite the interior surface, and the connecting threading extends along at least a portion of the interior surface of the anchor body, and wherein the proximal end of the elongated connecting member of the receiving body is inserted into the distal end of the anchor body with the connecting threading of the anchor body mated with the complimentary threading of the elongated connecting member.

24. The surgical anchor system of claim 23, wherein the elongated connecting member is an elongated pin that defines a major length and a cross-sectional width, and the major length is greater than the cross-sectional width.

25. The surgical anchor system of claim 23, wherein a portion of the receiving body defining the aperture is larger than a cross-sectional width of the lumen so as to prevent the receiving body from being inserted into the lumen of the fixation member.

26. The surgical anchor system of claim 18, wherein the anchor inserter includes at least two connection prongs extending from the distal end of the shaft, and the anchor body defines at least two channels interposed between the lumen and the exterior surface of the anchor body, the at least two channels opening towards the proximal end of the anchor body, and wherein the at least two connection prongs of the shaft are inserted into the at least two channels of the fixation member.

27. The surgical anchor system of claim 18, wherein the anchor inserter includes a threaded connector on the distal end of the rod, the elongated connecting member includes a corresponding threaded connector, and the rod extends through the lumen defined by the anchor body with the threaded connector of the rod mated with the corresponding threaded connector of the elongated connecting member.

28. The surgical anchor system of claim 18, wherein the fixation member includes a connecting groove or rib, the elongated connecting member of the receiving body includes a complimentary rib or groove that is configured to mate with the connecting groove or rib of the fixation member, and the fixation member is configured to move relative to the receiving body without rotating the shaft relative to the rod.

29. The surgical anchor system of claim 18, wherein the aperture is transverse to a major axis of the fixation member.

30. A method comprising:
inserting a surgical attachment element through an aperture defined by a receiving body, the receiving body including an elongated connecting member extending between a proximal end to a distal end, the aperture defined at the distal end of the elongated connecting member; and
moving a fixation member that includes an anchor body along the elongated connecting member so as to vary a distance between a distal end of the fixation member and the aperture of the receiving body.

31. The method of claim 30, wherein the fixation member is a fixation screw that includes exterior helical threading extending along at least a portion of an exterior surface of the anchor body.

32. The method of claim 30, wherein the fixation member is a fixation plug that includes exterior ribbing extending along at least a portion of an exterior surface of the anchor body.

33. The method of claim 30, further comprising connecting an anchor inserter that includes a shaft defining a lumen extending from a proximal end of the shaft to a distal end of the shaft, and a rod extending through the lumen defined by the shaft, wherein connecting the anchor inserter includes connecting the distal end of the shaft to the fixation member and connecting a distal end of the rod to the receiving body.

34. The method of claim 33, wherein the anchor inserter includes at least two connection prongs extending from the distal end of the shaft, and the anchor body of the fixation member defines a lumen extending from the proximal end through the distal end of the anchor body, the anchor body further defining at least two channels interposed between the lumen and an exterior surface of the anchor body, the at least two channels opening towards the proximal end of the anchor body, and wherein connecting the distal end of the shaft to the fixation member comprises inserting the at least two connection prongs of the shaft into the at least two channels of the fixation member.

35. The method of claim 33, wherein the anchor inserter includes a threaded connector on the distal end of the rod, the elongated connecting member of the receiving body includes a corresponding threaded connector, and wherein connecting the distal end of the rod to the elongated connecting member comprises screwing the threaded connector on the distal end of the rod into the elongated connecting member.

36. The method of claim 33, wherein the fixation member includes connecting threading, the elongated connecting member includes complimentary threading mated with the connecting threading of the fixation member, and wherein moving the fixation member along the elongated connecting member comprises rotating the shaft of the anchor inserter relative to the rod of the anchor inserter.

37. The method of claim 36, wherein the anchor body defines a lumen extending from a proximal end of the anchor body to a distal end of the anchor body, the anchor body defines an interior surface facing the lumen and an exterior surface opposite the interior surface, and the connecting threading extends along at least a portion of the interior surface of the anchor body, and wherein the proximal end of the elongated connecting member is inserted into the distal end of the anchor body with the connecting threading of the anchor body mated with the complimentary threading of the elongated connecting member.

38. The method of claim 30, wherein the aperture is transverse to a major axis of the fixation member.

39. The method of claim 30, further comprising inserting the fixation member into a hole defined in a bone so as to secure the surgical attachment element to the bone.

40. The method of claim 30, wherein inserting the surgical attachment element through the aperture comprises inserting a surgical mesh attachment device through the aperture.

41. A surgical anchor comprising:
receiving means for receiving a surgical attachment element; and
fixation means for securing the surgical attachment element to a bone hole;
wherein the receiving means are connected to the fixation means, and the fixation means are configured to move along an axis of the receiving means.
